(19) United States
(12) Patent Application Publication
(10) Pub. No.: US 2023/0001047 A1
(43) Pub. Date: Jan. 5, 2023

(54) Collagen gel formulations

(71) Applicants: THE UNIVERSITY OF SYDNEY, Sydney, NSW (AU); South East Sydney Local Health District, of the Sutherland Hospital and Community Health Service, Caringbah, NSW (AU)

(72) Inventors: Gerard SUTTON, Sydney, NSW (AU); Yihui SONG, Sydney, NSW (AU); Jingjing YOU, Sydney, NSW (AU)

(21) Appl. No.: 17/756,147

(22) PCT Filed: Nov. 18, 2020
(86) PCT No.: PCT/AU2020/051247

§ 371 Date: May 18, 2022

(30) Foreign Application Priority Data
Nov. 18, 2019 (AU) 2019904331

(51) Int. Cl. A61L 24/10; A61K 47/42

(52) U.S. Cl. CPC A61L 24/102; A61L 24/106; A61L 24/108; A61K 47/42; A61L 2300/428; A61L 2300/43; A61L 2300/414; A61L 2300/252; A61L 2300/418

(57) ABSTRACT

The present inventors have developed printable collagen bioinks using unmodified type I collagen with mechanical and structural properties that facilitate application to tissue in a structured form. In particular, the compositions of the present invention may be used to apply collagen gels to tissue (e.g. eye) using two- or three-dimensional (extrusion) bioprinting techniques.

### INCORPORATION BY CROSS-REFERENCE

This application claims priority from Australian provisional patent application number 2019904331, filed on 18 Nov. 2019, the entire contents of which are incorporated herein by cross-reference.

### TECHNICAL FIELD

The present invention relates generally to the fields of biology and medicine. More specifically, the present invention relates to compositions suitable for the delivery of agents to biological targets such as tissues and cells and/or capable of providing protection to said biological targets, and methods for the production thereof.

### BACKGROUND

When damage occurs to biological tissue, for example, as a result of accidental mechanical injury, surgery or disease, a major priority is to effectively seal the site of injury. Tissue sealants have a wide variety of applications including, but not limited to, the prevention of further injury, prevention of infection and minimisation of blood loss. Increasingly, tissue sealants are also being used to deliver agents to damaged tissue which may aid healing and/or help to prevent infection. Adverse effects such as allergic reactions are less likely when sealants are produced from naturally occurring materials.

Sealants vary widely in their mechanism of action and ease of application. An effective sealant flows freely during administration, but has the capacity to maintain shape/structure following application. The latter property is of particular importance when the shape of the biological tissue under repair is crucial to its function, for example, corneal tissue.

The cornea is the clear component of the protective covering of the eye. It allows light to pass through the pupil and is the primary refractive element of the eye's optical system. The cornea consists of five layers: the outer epithelium, Bowman's layer, the stroma, Descemet's membrane, and the inner endothelium. The corneal stroma accounts for approximately 90% of the overall thickness of the cornea and is mostly made up of collagen.

Corneal blindness is a major cause of blindness worldwide, second only to cataracts in terms of numbers. Causes of corneal blindness are diverse, and include diseases such as trachoma, onchocerciasis, leprosy, ophthalmia neonatorum, and xerophthalmia, and other processes such as ocular trauma, corneal ulceration, and complications arising from the use of traditional eye medicines.

Corneal injuries represent the most common ophthalmic emergency presentation in Australia and approximately 75% of all cases are due to the presence of foreign bodies or abrasions in the cornea. These injuries alone are estimated to cost the Australian population more than $155 million per year and, if not treated effectively, can lead to infection and scarring, resulting in permanently impaired vision.

In mild cases, a damaged cornea is able to regenerate via normal healing pathways. In other cases, however, the cornea's normal healing mechanism is insufficient, leading to the formation of non-healing defects which can result in corneal melting, corneal neovascularisation, loss of transparency, infection, scarring and diminished vision to the point of blindness.

Current medical treatments for corneal injuries include antibiotics, eye pads, sutures and surgical glues, which may help with minor issues. However, they do not adequately address issues arising in more advanced situations including pain relief, infection and/or the development of scar tissue. Infection represents a significant complication and often requires hospitalisation. Scarring, which is common in severe corneal injuries, can lead to permanent vision loss. In such cases, corneal transplantation is the only option for visual rehabilitation, but a shortage of donor corneas exists worldwide.

Many of the aforementioned issues are not restricted to corneal injuries, and also prevail in the case of damage to other body tissues.

A need exists for improved compositions and methods for sealing biological tissue, and/or for the effective delivery of agents to biological targets such as tissues and cells.

### SUMMARY OF THE INVENTION

The present invention alleviates at least one of the problems associated with current compositions and/or methods for sealing biological tissue and the delivery of agents to biological targets. In the context of the cornea, one of the main approaches currently is to reconstruct corneal defects with natural or synthetic materials. Collagen is the main natural material used, being the major protein in the cornea. Collagen contributes 60-80% of the dry weight of the cornea, and type I collagen is the predominant type in corneal stroma. However, the strength and elasticity of current naturally generated collagen products are limited. Chemical modification or cross-linking is normally required to improve the robustness of type I collagen and other types of collagen generated for therapeutic use. Biomechanical properties of current collagen-based products remain insufficient for ongoing use, as an irregular corneal shape is normally observed over time.

In addition to collagen implants, collagen sealant and bioinks have also been proposed and developed. However, the application of such bioinks has been limited by complicated printing methods and lack of transparency of the resulting products.

Instead of using collagen implants as stromal equivalents, the present inventors have developed printable collagen bioinks using unmodified type I collagen for printing directly onto the cornea. The bioinks of the present invention may also be printed directly onto a range of other biological targets (e.g. tissue, membranes, cells). The printed collagen bioinks can be solidified to form a temporary structure that will be degraded safely, and which allows the migration of surrounding corneal cells and restructuring of the stroma. The collagen bioinks of the present invention may be printed using two-dimensional or three-dimensional (extrusion) bioprinting techniques. Further, the bioinks of the present invention are able to be photo-crosslinked. The bioinks described herein may be transparent and adhesive and can support cell migration and proliferation. Bioactive molecules (e.g. growth factors) may be delivered using the biodegradable bioinks.

Without limitation, the compositions and methods described herein are generally useful for the delivery of agents (e.g. drugs and/or or other substances) to biological targets (e.g. tissue, membranes, cells) and may find application, for example, in the sealing of tissue, including corneal tis sue.

The present invention relates at least in part to the following embodiments:

Embodiment 1. A composition comprising:
3-15 mg/ml type I collagen;
0.135-0.5 M sodium ions and/or 0.008-0.4 M calcium ions; and
one or more crosslinking agents.

Embodiment 2. The composition of embodiment 1, wherein the composition comprises 0.01-0.5% (w/v) riboflavin or 0.01-0.5% (w/v) Rose Bengal.

Embodiment 3. The composition of embodiment 1 or embodiment 2, wherein the one or more crosslinking agents are capable of activation by light.

Embodiment 4. The composition of embodiment 3, wherein the light is UV light, blue light, green light or white light.

Embodiment 5. The composition of embodiment 1, wherein the composition comprises fibrinogen and/or thrombin.

Embodiment 6. The composition of embodiment 5, wherein the composition comprises 1.6-6 mg/ml fibrinogen.

Embodiment 7. The composition of embodiment 5 or embodiment 6, wherein the composition comprises 1-5 U/mL thrombin.

Embodiment 8. The composition of any one of embodiments 1 to 7, further comprising any one or more of: a culture medium, growth factors, hormones, matrix proteins, glycoproteins, vitamins, ions other than sodium ions or calcium ions, ion sources, fibronectin, amino acids, antibiotics, anaesthetics, factor XIII, Fetal Bovine Serum (FBS), human serum, platelet lysate, human platelet lysate.

Embodiment 9. The composition of embodiment 8, wherein the composition comprises a culture medium comprising the ions and amino acids.

Embodiment 10. The composition of embodiment 8 or embodiment 9, wherein:
(i) the growth factors comprise human epidermal growth factor (hEGF) and/or fibroblast growth factor (FGF); and/or
(ii) the vitamins comprise ascorbate (vitamin C); and/or
(iii) the matrix proteins comprise collagen IV; and/or
(iv) the hormones comprise insulin; and/or
(v) the glycoproteins comprise transferrin.

Embodiment 11. The composition of any one of embodiments 1 to 10, wherein the ions are components of an ionic salt included in the composition.

Embodiment 12. The composition of any one of embodiments 1 to 11, wherein the composition further comprises mammalian cells.

Embodiment 13. The composition of embodiment 12, wherein the mammalian cells comprise or consist of human cells.

Embodiment 14. The composition of any one of embodiments 1 to 13, wherein the type I collagen is neutralised.

Embodiment 15. The composition of any one of embodiments 1 to 14, wherein the composition comprises:

(i) 3-15 mg/ml type I collagen, 0.135-0.2 M sodium ions, and 0.01-0.05 M calcium ions;

or

(ii) 4-12 mg/ml type I collagen, 0.135-0.16 M sodium ions, and 0.015-0.03 M calcium ions; or

(iii) 5-11 mg/ml type I collagen, 0.135-0.14 M sodium ions, and 0.018-0.02 M calcium ions.

Embodiment 16. The composition of any one of embodiments 1 to 15, wherein the composition comprises:

(i) less than 15 mg/ml type I collagen;

(ii) more than 0.135 M sodium ions; and

(iii) more than 0.018 M calcium ions.

Embodiment 17. A method of preparing a composition, the method comprising:

(i) providing a solution comprising:
3-15 mg/ml type I collagen;
one or more crosslinking agents; and
0.135-0.5 M sodium ions and/or 0.008-0.4 M calcium ions;

(ii) applying the solution to a surface; and

(iii) applying light capable of activating the one or more crosslinking agents to the solution.

Embodiment 18. The method of embodiment 17, wherein applying the solution to a surface forms a layer, and wherein steps (ii) and (iii) are repeated a plurality of times, wherein each layer is applied on top of the preceding layer.

Embodiment 19. The method of embodiment 17 or embodiment 18, wherein the one or more crosslinking agents comprise 0.01-0.5% (w/v) riboflavin or 0.01-0.5% (w/v) Rose Bengal, and wherein the light comprises UV light, blue light, green light or white light.

Embodiment 20. The method of any one of embodiments 17 to 19, wherein:

(a) the one or more crosslinking agents are combined with a base to form part A; (b) the type I collagen is combined with the sodium ions and/or calcium ions to form part B; and

(c) parts A and B are mixed to form a solution prior to step (iii).

Embodiment 21. A method of preparing a composition, the method comprising:

(i) providing a solution comprising:
3-15 mg/ml type I collagen;
one or more crosslinking agents; and
0.135-0.5 M sodium ions and/or 0.008-0.4 M calcium ions;

(ii) applying the solution to a surface, wherein the solution is divided into at least two components prior to applying to the surface.

Embodiment 22. The method of embodiment 21, further comprising the steps of:

(iii) adding fibrinogen to at least one component to form formulation (a);

(iv) adding thrombin to at least one component to form formulation (b); and

(v) combining formulations (a) and (b) to form a gel.

Embodiment 23. The method of embodiment 22, wherein:
1.6-6 mg/ml fibrinogen is added to at least one component to form formulation (a) in step (iii); and/or
1.5 U/mL thrombin is added to at least one component to form formulation (b) in step (iv).

Embodiment 24. The method of any one of embodiments 17 to 23, wherein the solution further comprises any one or more of: a culture medium, growth factors, hormones, matrix proteins, glycoproteins, vitamins, ions other than sodium ions or calcium ions, ion sources, fibronectin, amino acids, antibiotics, anaesthetics, factor XIII, Fetal Bovine Serum (FBS), human serum, platelet lysate, human platelet lysate.

Embodiment 25. The method of embodiment 24, wherein the solution comprises a culture medium comprising the ions and amino acids.

Embodiment 26. The method of embodiment 24 or embodiment 25, wherein:
(i) the growth factors comprise human epidermal growth factor (hEGF) and/or fibroblast growth factor (FGF); and/or
(ii) the vitamins comprise ascorbate (vitamin C); and/or
(iii) the matrix proteins comprise collagen IV; and/or
(iv) the hormones comprise insulin; and/or
(v) the glycoproteins comprise transferrin.

Embodiment 27. The method of any one of embodiments 17 to 26, wherein the ions are components of an ionic salt included in the solution.

Embodiment 28. The method of any one of embodiments 17 to 27, wherein the solution further comprises mammalian cells.

Embodiment 29. The method of embodiment 28, wherein the mammalian cells comprise or consist of human cells.

Embodiment 30. The method of any one of embodiments 17 to 29, wherein the type I collagen is neutralised.

Embodiment 31. A composition obtained or obtainable by the method of any one of embodiments 17 to 30.

Embodiment 32. A method of sealing the surface of tissue, the method comprising applying the composition of any one of embodiments 1 to 16 or embodiment 31 to the tissue.

Embodiment 33. A method of delivering agents to tissue, the method comprising applying the composition of any one of embodiments 1 to 16 or embodiment 31 to the tissue.

Embodiment 34. A composition of any one of embodiments 1 to 16 or embodiment 31 for use in sealing the surface of tissue.

Embodiment 35. A composition of any one of embodiments 1 to 16 or embodiment 31 for use in delivering agents to tissue.

Embodiment 36. Use of a kit, package or device comprising type I collagen, sodium ions, calcium ions, and one or more crosslinking agents, for preparing a composition comprising:
3-15 mg/ml of the type I collagen;
0.135-0.5 M of the sodium ions and/or 0.008-0.4 M of the calcium ions; and
the one or more crosslinking agents.

Embodiment 37. Use of the kit, package or device of embodiment 36, wherein the composition comprises 0.01-0.5% (w/v) riboflavin or 0.01-0.5% (w/v) Rose Bengal.

Embodiment 38. Use of the kit, package or device of embodiment 36 or embodiment 37, wherein the one or more crosslinking agents are capable of activation by light.

Embodiment 39. Use of the kit, package or device of embodiment 38, wherein the light comprises UV light, blue light, green light or white light.

Embodiment 40. Use of the kit, package or device of embodiment 36, wherein the composition comprises fibrinogen and thrombin, and wherein
the fibrinogen is present in a first compartment;
the thrombin is present in a second compartment; and wherein
the kit, package or device is configured to allow separation of the fibrinogen of the first compartment and the thrombin of the second compartment during and following loading of the fibrinogen and thrombin into the kit, package or device, and wherein the kit, package or device further comprises a means to facilitate mixing of the fibrinogen of the first compartment with the thrombin of the second compartment.

Embodiment 41. Use of the kit, package or device of embodiment 40, wherein the composition comprises:
1.6-6 mg/ml fibrinogen; and/or
1-5 U/mL thrombin.

Embodiment 42. Use of the kit, package or device of any one of embodiments 36 to 41, wherein the composition further comprises any one or more of: a culture medium, growth factors, hormones, matrix proteins, glycoproteins, vitamins, ions other than sodium ions or calcium ions, ion sources, fibronectin, amino acids, antibiotics, anaesthetics, factor XIII, Fetal Bovine Serum (FBS), human serum, platelet lysate, human platelet lysate.

Embodiment 43. Use of the kit, package or device of any one of embodiments 36 to 42, wherein any one or more of the type I collagen, sodium ions, calcium ions and/or one or more crosslinking agents is separated from other component/s within the kit.

Embodiment 44. The composition of any one of embodiments 1 to 16 or embodiment 31, wherein the composition is transparent.

Embodiment 45. The composition of any one of embodiments 1 to 16 or embodiment 31, wherein the composition has the capacity to maintain or substantially maintain shape/structure following printing.

### Definitions

As used in this application, the singular form “a”, “an” and “the” include plural references unless the context clearly dictates otherwise. For example, the term “component” also includes a plurality of the components.

As used herein, the term “comprising” means “including”. Variations of the word “comprising”, such as “comprise” and “comprises,” have correspondingly varied meanings. Thus, for example, a composition “comprising” component ‘A’ may consist exclusively of component ‘A’ or may include one or more additional components (e.g. component 13′ and/or component ‘C’).

As used herein, the term “subject” includes any animal of economic, social or research importance including bovine, equine, ovine, primate, avian and rodent species. Hence, a “subject” may be a mammal such as, for example, a human, or a non-human mammal.

As used herein, the term “tissue” will be understood to encompass both cells that are component/s of the tissue and organ/s formed from the tissue.

As used herein, the term “kit” refers to any delivery system for delivering materials. Such delivery systems include systems that allow for the storage, transport, or delivery of reaction reagents (e.g., labels, reference samples, supporting material, etc. in appropriate containers) and/or supporting materials (e.g., buffers, written instructions for performing an assay etc.) from one location to another. For example, kits may include one or more enclosures, such as boxes, containing the relevant reaction reagents and/or supporting materials. The term “kit” includes both fragmented and combined kits. A “fragmented kit” refers to a delivery system comprising two or more separate containers that each contains a sub-portion of the total kit components. The containers may be delivered to the intended recipient together or separately. Any delivery system comprising two or more separate containers that each contains a sub-portion of the total kit components are included within the meaning of the term “fragmented kit”. A “combined kit” refers to a delivery system containing all of the components of a reaction assay in a single container (e.g. in a single box housing each of the desired components).

As used herein, the term “about” when used in reference to a recited numerical value includes the recited numerical value and numerical values within plus or minus ten percent of the recited value.

As used herein, the term “plurality” means more than one. In certain specific aspects or embodiments, a plurality may mean 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, or more, and any numerical value derivable therein, and any range derivable therein.

As used herein, the term “between” when used in reference to a range of numerical values encompasses the numerical values at each endpoint of the range. For example, sodium ions with a concentration of between 0.135 M and 3 M is inclusive of sodium ions with a concentration 0.135 M and sodium ions with a concentration 3 M.

As used herein, the term “more than” when used in reference to a numerical value will be understood to mean “greater than or equal to”. For example, more than 0.135 M sodium ions encompasses a concentration of 0.135 M sodium ions and all concentrations of sodium ions greater than 0.135 M.

As used herein, the term “less than” when used in reference to a numerical value will be understood to mean “less than or equal to”. For example, less than 15 mg/ml type I collagen encompasses a concentration of 15 mg/ml type I collagen and all concentrations of type I collagen less than 15 mg/ml.

As used herein, the term “neutralised” when used to describe type I collagen will be understood to mean that the pH of the collagen solution is between 6.7 and 7.6. For example, “neutralised” type I collagen could have a pH between 6.8 and 7.5, or between 6.9 and 7.4, or between 7.0 and 7.3, or between 6.9 and 7.2, etc.

Any description of prior art documents herein, or statements herein derived from or based on those documents, is not an admission that the documents or derived statements are part of the common general knowledge of the relevant art.

For the purposes of description, all documents referred to herein are hereby incorporated by reference in their entirety unless otherwise stated.

### BRIEF DESCRIPTION OF THE FIGURES

Preferred embodiments of the present invention will now be described by way of example only, with reference to the accompanying figures wherein:

FIG. 1 shows the results of testing the effect of ions on the stability of neutralised collagen I solutions. For each solution generated, 90 parts of collagen solution were neutralised with 2.7 parts of 5 M NaOH and then mixed with 7.3 parts of various salt solutions. The solutions tested included 1×Phosphate buffer saline ((PBS) consisting of NaCl, KCl, Na₂HPO₄ and KH₂PO₄), NaCl, KCl, Na₂HPO₄, KH₂PO₄, and sodium ascorbate (NaC₆H₇NO₆). The solutions were left on the bench for 1 hr, 1 day, and 1 week prior to examination for precipitation formation. The composition of 1×PBS (unit: mM) was Na⁺:156.9, K⁺: 109.8, Cl⁻: 244.9, HPO₄²⁻: 10, H₂PO⁴⁻: 1.8. FIG. 1 shows that the inclusion of NaCl (at minimal concentration of 68.4 mM) or CaCl₂ (with a minimum concentration of 18 mM) was optimal to make up the soluble transparent collagen gels.

FIG. 2. Riboflavin powder dissolved in CaCl₂/PBS. FIG. 2a shows riboflavin in CaCl₂ before centrifugation. Left tube: 0.1% riboflavin solution; Right tube: 0.2% riboflavin solution. FIG. 2b shows riboflavin in CaCal₂ after centrifugation. Left tube: 0.1% riboflavin solution remained transparent. Right tube: 0.2% riboflavin precipitated in CaCl₂ solution as indicated by the arrow.

FIG. 3 provides representative images of crosslinked collagen ink on glass slides. FIG. 3a shows collagen ink crosslinked by a tissue culture hood UV lamp. FIG. 23 shows collagen ink crosslinked by 3 mw/cm² 365 nm UV. FIG. 3c shows collagen ink crosslinked by 470 nm 10 mw/cm² blue light.

FIG. 4 provides representative images of human collagen-based collagen ink on glass slides crosslinked by 3 mw/cm² 365 nm UV.

FIG. 5 provides a representative image of collagen bioink crosslinked by Rose Bengal—green light.

FIG. 6 is a series of graphs demonstrating the photorheology properties of collagen bioink samples. FIGS. 6a to 6e show the changes in storage modulus and loss modulus over time following the application of UV light to the samples. FIG. 6a shows 12 mg/ml collagen with PBS. FIG. 6b shows 12 mg/ml collagen with calcium ions. FIG. 6c shows 6 mg/ml collagen with calcium ions. FIG. 6d shows 3 mg/ml collagen with calcium ions. FIG. 6e shows 12 mg/ml collagen with calcium ions, stored in a −30° C. freezer.

FIG. 7 provides representative images of freshly made 12 mg/ml collagen bioink after crosslinking (FIG. 7a) and −30° C.-stored 12 mg/ml collagen bioink after crosslinking (FIG. 7b).

FIG. 8 is a graph plotting the viscosity of a 6 mg/ml riboflavin-added collagen bioink containing 18 mM Ca²⁺ on the y axis versus increasing shear rate on the x axis. The shear trimming behaviour displayed is required for extrusion 3D printing.

FIG. 9 is a graph showing the storage/loss modulus of sodium ascorbate-incorporated collagen bioink and sodium chloride-incorporated collagen bioink over time following UV treatment. Both bioinks had the same collagen concentration. The graph shows that the strength of photo-crosslinking initiated by UV irradiation was halved with the addition of ascorbic acid.

FIG. 10 provides representative images of a folded collagen gel (FIG. 10a) and a collagen gel unfolded in water (FIG. 10b).

FIG. 11 shows the total transmittance of the collagen gel in the 400-700 nm wavelength range.

FIG. 12 provides representative images of structures formed by line stacking of collagen bioinks. FIG. 12a provides images of a structure formed by 6 mg/ml collagen bioink and manipulating the structure with tweezers. FIG. 12b shows a structure formed by line stacking of 12 mg/ml collagen ink and manipulating the structure with tweezers.

FIG. 13 provides representative images of HCET cells on top of the crosslinked collagen bioink (FIG. 13a) and the DAPI staining result of one section of the collagen gel (FIG. 13b).

FIG. 14 provides representative images of HCSCs on top of the crosslinked collagen bioink (FIG. 14a) and degradation of the collagen gel with HCSCs seeded on top (FIG. 14b).

FIG. 15 provides representative images of cells encapsulated in a crosslinked collagen bioink on day 1 (FIG. 15a) and cells encapsulated in a crosslinked collagen bioink on day 5 (FIG. 15b).

FIG. 16 provides the results of a cell delivery experiment using hCSCs. The images show collagen bioink and cells 1 day (FIG. 16a), 3 days (FIG. 16b) and 7 days (FIG. 16c) after crosslinking.

FIG. 17 shows a comparison of BrdU readings of HCET cells in a 96 well plate, on top of the collagen gel (left)/on a tissue culture plate (right) (p=0.04).

FIG. 18 shows a comparison of BrdU readings of hCSCs cells in a 96 well plate, on top of the collagen gel (left)/on a tissue culture plate (right) (p=0.01).

FIG. 19 provides a representative image of immortalized human corneal endothelial cells on top of crosslinked collagen IV-incorporated 6 mg/ml collagen bioink. ink. The cells reached confluence in 7 days

FIG. 20 shows a comparison of BrdU readings of corneal stromal cells in a 96 well plate, on top of FBS-incorporated collagen gel (left)/without FBS-incorporated collagen gel (right) (p=0.04).

FIG. 21 shows the filling of a porcine cornea with the compositions of the present invention. In FIG. 21a, the hole was created using a 2 mm trephine. In FIG. 21b, the hole was filled with 6 mg/ml collagen bioink after rinsing.

FIG. 22 provides images of filling gaps created in porcine cornea (a) the gap created using a 2 mm trephine (b) the gap filled with 12 mg/ml collagen ink.

FIG. 23 provides images of filling gaps created in porcine cornea (a) the gap created using a 4 mm trephine (b) the gap filled with collagen ink.

FIG. 24 provides a non-limiting example of an TOP simulating system with a UV curing device.

FIG. 25 provides images showing porcine corneal sealing. FIG. 25a shows a 6 mg/ml collagen bioink sealing a corneal perforation. In FIGS. 25b and 25c, 12 mg/ml collagen bioink failed to adhere.

FIG. 26 provides images showing porcine corneal sealing. (a) 1.5 mm diameter perforation; (b) a 12 mg/ml collagen bioink seals the gap.

FIG. 27 provides images showing porcine corneal sealing. (a) 2 mm diameter perforation; (b) a 12 mg/ml collagen bioink seals the gap.

FIG. 28 provides a graph showing the relationship of the volumes of parts A and B in crosslinkable collagen ink.

FIG. 29 provides images of thin collagen films generated.

FIG. 30 provides an image of a thicker collagen structure prior to rinsing.

FIG. 31 provides an image showing the pores of a 3D printed mesh structure.

FIG. 32 provides an image showing Calcein-AM staining of a cell-loaded structure 2 weeks post printing.

### DETAILED DESCRIPTION

The present inventors have developed printable collagen bioinks using unmodified type I collagen with mechanical and structural properties that facilitate application to tissue in a structured form. In particular, the compositions of the present invention may be used to apply collagen gels to tissue (e.g. eye) using two- or three-dimensional (extrusion) bioprinting techniques. The compositions provide a means of delivering agents to biological targets (e.g. organs, tissues, cells). While suitable for application to the cornea, the compositions described herein provide a platform for numerous applications in the areas of sealing tissue and the delivery of agents by virtue of providing, for example, structural support, viable cells, and other factors.

There is a need in the art for effective collagen-derived sealants and adhesives for surfaces such as ocular surfaces and the corneal stroma. The compositions described herein are based on natural type I collagen which, in the context of the cornea, can reconstitute the major protein in this tissue. The compositions of the present invention are also ideal agents for the delivery of a diverse range of growth factors and other agents. The compositions described herein may utilise biomaterial that mimics in vivo tissue and acts as a scaffold for cells to populate, and/or, through the manipulation of conditions, encourages the cells themselves to regenerate their surrounding matrix. In the context of their suitability for application to eye tissue the present inventors have, for example, addressed the difficulties of creating a matrix that can embody the structural integrity of the tissue under treatment (e.g. cornea) whilst maintaining transparency and still being porous and biocompatible enough to allow for the infiltration, migration and/or proliferation of corneal cells and growth factors.

The balance between providing the nutritional needs of damaged tissue while meeting the structural, mechanical and physical requirements of damaged tissue (e.g. eye tissue such as cornea) was a problem existing at the time that the present invention arose. The present invention provides improved compositions and methods for delivering agents to a broad variety of biological targets. Without limitation to any particular application, the compositions may be used for sealing tissue and the delivery of agents to biological targets.

### Compositions for Delivery of Biological Agents

The present invention provides compositions suitable for the delivery of agents to biological targets such as tissues and cells. The compositions may also be used as sealants and/or adhesives for said biological targets.

The compositions utilise a base scaffold material to provide structural support upon application to a biological target (e.g. tissues, membranes, cells, organs), to facilitate the delivery of agents to the biological target.

No particular limitation exists regarding the specific material/s used to generate the scaffolds.

For example, the scaffolds may be collagen scaffolds. These may be generated, for example, via the use of type I collagen in the compositions. The type I collagen used may be unmodified when compared to its naturally occurring counterpart.

The compositions of the present invention may further optionally comprise ions and/or one or more sources of ions. Non-limiting examples of suitable ions include calcium ions and sodium ions. Non-limiting examples of suitable ion sources include compounds comprising calcium (e.g. calcium chloride) and sodium (e.g. sodium chloride). Calcium ions and sodium ions may be present in the compositions of the present invention together or individually.

The present inventors have identified optimal relative concentrations of type I collagen, sodium ions and/or calcium ions for the compositions of the present invention, some of which are described in the Examples and claims of the present application. It will be understood that the relative concentrations of type I collagen, sodium ions and/or calcium ions disclosed are exemplary only.

The composition may comprise 1-20 mg/ml type I collagen, 0.07-0.5 M sodium ions and/or 0.008-0.4 M calcium ions. In some embodiments, the composition may comprise 1-20 mg/ml type I collagen, 0.07-0.3 M sodium ions and/or 0.008-0.1 M calcium ions. In some embodiments, the composition may comprise 3-15 mg/ml type I collagen, 0.135-0.3 M sodium ions and/or 0.008-0.1 M calcium ions. In some embodiments, the composition may comprise 3-15 mg/ml type I collagen, 0.135-0.2 M sodium ions and/or 0.01-0.05 M calcium ions. In some embodiments, the composition may comprise 4-12 mg/ml type I collagen, 0.135-0.16 M sodium ions and/or 0.015-0.03 M calcium ions. In some embodiments, the composition may comprise 5-10 mg/ml type I collagen, 0.135-0.14 M sodium ions and/or 0.018-0.02 M calcium ions. In some embodiments, the composition may comprise less than 15 mg/ml type I collagen and more than 0.135 M sodium ions and/or more than 0.018 M calcium ions.

The compositions of the present invention may further comprise one or more crosslinking agents. In some embodiments of the invention, the crosslinking agent is riboflavin. The riboflavin could be present at a concentration of 0.01-0.5% (w/v). Light, such as UV light or blue light could be used to activate the riboflavin, crosslinking the composition. The person skilled in the art will be aware of other suitable photo-crosslinking agents and light sources, for example, Rose Bengal dye and green light, both of which have been approved for several applications to the cornea. In some embodiments, Rose Bengal is used as a photo-crosslinking agent and is activated by green light. In some embodiments, Rose Bengal is used as a photo-crosslinking agent and is activated by white light. In some embodiments, 0.01-0.5% (w/v) Rose Bengal is used for photo-crosslinking. In some embodiments, compositions provided by crosslinking the collagen bioink with Rose Bengal and a suitable light source will be coloured. In some embodiments, the colour will be pink. These coloured compositions may be used for monitoring collagen metabolism in tissue, or for other uses requiring tracking of collagen activity.

In some embodiments of the present invention, the crosslinking agents are fibrinogen and/or thrombin. Concentrations used may be 1.6-6 mg/ml fibrinogen and 1-5 U/mL thrombin. Concentrations used may be 0.1-20 mg/ml fibrinogen and 2-20 U/mL thrombin. The present inventors have identified optimal relative concentrations of fibrinogen and thrombin for the compositions of the present invention which are described in the claims of the present application. It will be understood that the relative concentrations of fibrinogen and thrombin disclosed are exemplary only.

The compositions of the present invention may include platelet lysate. The platelet lysate may, for example, be mammalian platelet lysate (e.g. generated using human, canine, feline, bovine, porcine, equine, caprine, hircine, murine, leporine, cricetine, or musteline platelets, or any combination thereof). The source of platelets utilised to generate the platelet lysate will generally depend on the specific purpose for which the composition is to be used. As known to those of ordinary skill in the art, platelet lysate is generated by isolating platelets, lysing them and removing cellular debris. The constituents of platelet lysate and its applications have been well analysed (see, for example, Burnouf et al. Biomaterials. 2016 January; 76:371-87).

In some embodiments, the compositions do not comprise anticoagulants or are substantially free of anticoagulants which may be present at only trace amounts. Non-limiting examples of such anticoagulants include heparin, Vitamin K Antagonists (e.g. Warfarin, Coumarins), Rivaroxaban, Edoxaban, Apixaban, Dabigatran, and the like. In some embodiments, the platelet lysate comprises less than: 10% (v/v), 9% (v/v), 8% (v/v), 7% (v/v), 6% (v/v), 5% (v/v), 4% (v/v), 3% (v/v), 2% (v/v), 1% (v/v), 0.5% (v/v), anticoagulants.

Compositions according to the present invention may include cells. The cells may, for example, be mammalian cells (e.g. human cells, canine cells, feline cells, bovine cells, porcine cells, equine cells, caprine cells, hircine cells, murine cells, leporine cells, cricetine cells, musteline cells, or any combination thereof). The type of cells utilised will generally depend on the specific purpose for which the composition is to be used. For example, the cells may be of the same type as a tissue to which the composition is to be administered (e.g. eye surface cells including those of the central and/or peripheral corneal epithelium, bulbar and/or tarsal conjunctival epithelia, tarsal conjunctival stroma, and/or lid margin; skin cells including but not limited to keratinocytes, melanocytes, Merkel cells, and Langerhans cells; and neural tissue cells including but not limited to neurons and glial cells). Other examples include epithelial cells, keratocytes, neuronal cells, and endothelial cells. In some embodiments, the cells may be hematopoietic stem cells, bone marrow stem cells, neural stem cells, epithelial stem cells, skin stem cells, muscle stem cells, adipose stem cells, pluripotent stem cells, induced pluripotent stem cells, embryonic stem cells, mesenchymal stem cells, or any combination thereof. In some embodiments, the cells may be neuronal cells.

The platelet lysates and/or cells of the compositions may be autologous (i.e. self-derived from a given subject intended to receive the composition, or allogeneic (i.e. donor-derived).

The compositions of the present invention may comprise essential and/or non-essential amino acids. Non-limiting examples of suitable essential amino acids include isoleucine, leucine, lysine, methionine, phenylalanine, threonine, tryptophan, valine, cysteine, tyrosine, histidine and arginine.

The compositions of the present invention may comprise additional components (e.g. agent/s) including, but not limited to, fibronectin, anaesthetics, antibiotics, hormones (e.g. insulin), growth factors (e.g. human epidermal growth factor (hEGF), platelet derived growth factor, vascular endothelial growth factor, fibroblast growth factor (FGF), epithelial growth factor, transforming growth factor [including beta], and connective tissue growth factor), fibrin stabilizing factors (e.g. factor XIII), matrix protein/s (e.g. collagen [such as collagen IV], laminin, integrin), vitamins (e.g. vitamin C), glycoproteins (e.g. transferrin), Fetal Bovine Serum (FBS), human serum, platelet lysate, human platelet lysate and any combination thereof. In some embodiments, the composition comprises a culture medium comprising the ions and amino acids.

The compositions of the present invention may include other suitable ingredients including water and/or culture medium (e.g. DMEM, DMEM/F-12, MEM, CnT-PR). The culture medium may comprise, for example, any one or more of Glycine, L-Alanine, L-Arginine hydrochloride, L-Asparagine-H₂O, L-Aspartic acid, L-Cysteine hydrochloride-H₂O, L-Cystine 2HCl, L-Glutamic Acid, L-Glutamine, L-Histidine hydrochloride-H₂O, L-Isoleucine, L-Leucine, L-Lysine hydrochloride, L-Methionine, L-Phenylalanine, L-Proline, L-Serine, L-Threonine, L-Tryptophan, L-Tyrosine disodium salt dihydrate, L-Valine, Vitamins, Biotin, Choline chloride, D-Calcium pantothenate, Folic Acid, Niacinamide Pyridoxine hydrochloride, Riboflavin, Thiamine hydrochloride, Vitamin B12, i-Inositol, Inorganic Salts, Calcium Chloride (CaCl₂) (anhyd.), Cupric sulfate (CuSO₄-5H₂O), Ferric Nitrate (Fe(NO₃)₃″9H₂O), Ferric sulfate (FeSO₄-7H₂O), Magnesium Chloride (anhydrous), Magnesium Sulfate (MgSO₄) (anhyd.), Potassium Chloride (KCl), Sodium Bicarbonate (NaHCO₃), Sodium Chloride (NaCl), Sodium Phosphate dibasic (Na₂HPO₄) anhydrous, Sodium Phosphate monobasic (NaH₂PO₄—, Zinc sulfate (ZnSO₄-7H₂O), Other Components, D-Glucose (Dextrose), Hypoxanthine Na, Linoleic Acid, Lipoic Acid, Putrescine 2HCl, Sodium Pyruvate, Thymidine, or any combination thereof. In some embodiments, ions are provided as components of an ionic salt included in the composition.

In some embodiments of the invention, the compositions are transparent. Additionally or alternatively, the compositions may have the capacity to maintain or substantially maintain shape/structure following printing.

Non-limiting properties of the compositions include one or more of the following:

Non-Newtonian shear-thinning fluid properties, whereby the viscosity of the composition may decrease as the shear-rate increases. In some embodiments, the viscosity of the compositions may be in the range of 0.01 and 1000 Pa·s at room temperature.

Optical clarity without impeding or without substantially impeding vision arising from transmittance of light, for example, over 90% in the visual colour range of 400-700 nm.

Suitability for 2D and/or 3D printing (e.g. bioprinting/extrusion printing) with capacity to maintain or substantially maintain shape/structure following printing.

Suitability for printing while maintaining the viability of cells within the composition during the printing process.

Capacity to be provided in two- or three-dimensional structure with or without the inclusion of viable cells.

Capacity to sustain and/or promote the growth of cells (e.g. sustain and/or promote the expansion growth of primary human cells such as epithelial cells, keratocytes, neuronal cells, and endothelial cells).

Capacity to promote the formation of spheroid organoids.

Capacity for degradation by cells over time (e.g. 2-7 days).

Maintenance of cell viability over time (e.g. 7 days at 34° C.).

Capacity to adhere to various surfaces, including tissues, organs, membranes (e.g. mammalian and human tissues, organs, membranes).

### Preparation of Compositions

In general, the compositions of the present invention may be prepared by combining a plurality of different preparations. Lyophilised bovine collagen type I may be used in the preparation of the compositions. Additionally or alternatively, human collagen may be used. The type I collagen may be neutralised prior to the addition of ions and other components of the compositions. In some embodiments of the invention, the one or more crosslinking agents are combined with a base to form part A the type I collagen is combined with the ions, for example, sodium ions and/or calcium ions to form part B; and parts A and B are mixed to form a solution prior to applying the solution to a surface. In some embodiments, parts A and B are mixed in a ratio of between 1 and 20: between 200 and 300. In further embodiments, the ratio is 9:250. A person skilled in the art would recognise that various protocols could be used to prepare the compositions of the invention and that various buffer solutions could be used to keep the collagen at physiological pH and to maintain solubility.

In some embodiments of the present invention, the crosslinking agents are fibrinogen and thrombin. In some embodiments, a type I collagen preparation comprising thrombin may be maintained separately from a type I collagen preparation comprising fibrinogen, and the two preparations may be combined prior to or during application of the composition.

In some embodiments, the type I collagen plus fibrinogen and type I collagen plus thrombin preparations can be provided by establishing individual flow streams of the two separated components. These streams can be maintained in a state of continual flow for a suitable time period and be oriented to mix with each other at a given point to thereby provide a further stream of mixed components that is deposited on the biological target. Alternatively, the streams may be oriented to mix with each other at or on a surface of the biological target to which the composition is applied. Compositions of the present invention using fibrinogen and thrombin as crosslinking agents may not be transparent without the addition of platelet lysate. Such non-transparent collagen bioinks may be used for wider application than in ophthalmology.

Compositions of the present invention may comprise platelet lysate (e.g. mammalian platelet lysate, human platelet lysate). The platelet lysate may be prepared by any suitable method (e.g. lysing by freeze/thawing; see, for example, Chou and Burnouf, ISBT Science Series, Vol 12, Issue 1, February 2017, pages 168-175). The addition of platelet lysate to compositions of the present invention crosslinked by fibrinogen and thrombin may cause the compositions to become transparent.

Additionally, the present inventors have observed that the use of anticoagulants during platelet lysate preparation (e.g. when culturing) can have an impact on the capacity of platelet lysates to form compositions according to the present invention. Accordingly, in some embodiments of the present invention, the platelet lysates utilised are prepared without anticoagulants (e.g. heparin) at some or all stages of the preparation method.

In some embodiments, the invention provides devices and/or kits that facilitate the separation of different preparations needed to form the compositions of the invention until use. In some embodiments, the devices and kits may comprise at least two physically separated compartments, a first comprising a preparation of collagen solution with ions and thrombin and a second comprising a preparation of collagen solution with ions and fibrinogen. In some embodiments, either or both compartments may comprise additional components to be used in generating the composition (e.g. platelet lysate, ions, amino acids, cells, antibiotics, growth factors, vitamins, fibrin stabilising factors, anaesthetics and so on).

The devices and kits may further comprise a component providing a means to facilitate mixing of the two compartmentalised preparations such as, for example, by removal of barrier/s separating the first and second compartments, and/or by puncturing a seal or wall of either or both compartments. The skilled person will readily understand that various arrangements can be made for this purpose.

Additionally or alternatively, devices and kits may be configured in a manner that ensures mixing of the two compartmentalised preparations during or following release of the preparations from the device or kit.

In some embodiments, the devices and kits may comprise additional compartments comprising additional components to be used in generating the composition (e.g. platelet lysate, ions, amino acids, cells, antibiotics, growth factors, fibrin stabilizing factors, anaesthetics and so on). The device or kit may be configured in such a way to facilitate mixing of these additional components with each other and/or with the preparation/s of fibrinogen and/or thrombin during use of the device or kit.

The devices and kits may facilitate mixing of separated components prior to, during or immediately following discharge of the components from the device or kit.

In some embodiments, the compositions are bioinks and the device is a three-dimensional (3D) printer (e.g. an extrusion printer).

In some embodiments of the present invention, the crosslinking agent is riboflavin. In some embodiments, the riboflavin may be activated by UV light or blue light. The solution, which may contain type I collagen and sodium and/or calcium ions, may be extruded in a line and UV light or blue light may be applied. Additional lines may be applied on top of the first line to form a structure which will be crosslinked by the crosslinking agent and the application of light.

In some embodiments, photo-crosslinking occurs in under 15 minutes, under 14 minutes, under 13 minutes, under 12 minutes, under 11 minutes, under 10 minutes, under 9 minutes, under 8 minutes, under 7 minutes, under 6 minutes, under 5 minutes, under 4 minutes, under 3 minutes, under 2 minutes or under 1 minute. The light source used may be 3 mW/cm² 365 nm UV, 10 mW/cm² blue light or a tissue culture hood UV lamp.

In some embodiments, the photo-crosslinking agent Rose Bengal. In some embodiments, Rose Bengal is activated by green light. In some embodiments, Rose Bengal is activated by white light. In some embodiments, photo-crosslinking occurs in under 15 minutes, under 14 minutes, under 13 minutes, under 12 minutes, under 11 minutes, under 10 minutes, under 9 minutes, under 8 minutes, under 7 minutes, under 6 minutes, under 5 minutes, under 4 minutes, under 3 minutes, under 2 minutes or under 1 minute. The light source used may be 100 mw/cm² white light. The person skilled in the art would realise that the light source and parameters could be varied according to the particular application.

The collagen gels of the present invention may have various thicknesses according to the application, for example, the thickness of a gel could be between 50 μm to 5 mm.

### Applications of the Compositions

The present inventors have developed a composition for the delivery of agent/s to target tissues and cells with characteristics making it highly suitable for bioprinting. The compositions of the present invention may be used in applications where there is a need for delivery of agents (e.g. natural growth factors, drugs, nanoparticles, and/or cells,) and/or for the fixation of individual biological surfaces, and/or in tissue culture methods.

In some embodiments the compositions can act as tissue sealants and/or as a fixative for biological structures. They may provide structural and/or nutritional support to tissue. Additionally or alternatively, the compositions may facilitate the growth of target cell type/s, including those which may be provided as a component of the compositions and/or cells present in the target tissue.

In some embodiments, compositions provided by crosslinking the collagen bioink with Rose Bengal and a suitable light source will be coloured. In some embodiments, the collagen compositions may be pink. These coloured compositions may be used for monitoring collagen metabolism in tissue, or for other uses requiring tracking of collagen activity.

While no limitation exists as to the type of tissue to which the compositions may be applied, the present inventors have demonstrated that the compositions are effective in the sealing of eye tis sue.

For example, the compositions are demonstrated herein to be effective in sealing corneal tissue. In these embodiments, the compositions may be used to promote the proliferation and/or migration of corneal epithelial cells. The compositions may, for example, support multidirectional growth and/or stratification of corneal epithelial cells, which may partially or completely biodegrade the composition once a cell monolayer is formed.

The present invention thus provides methods for sealing tissue and for the delivery of a variety of agents to biological targets. The targets may, for example, be located in or around eye tissue including tissue of the central and/or peripheral corneal epithelium, bulbar and/or tarsal conjunctival epithelia, tarsal conjunctival stroma, and/or lid margin.

It will be appreciated by persons of ordinary skill in the art that numerous variations and/or modifications can be made to the present invention as disclosed in the specific embodiments without departing from the spirit or scope of the present invention as broadly described. The present embodiments are, therefore, to be considered in all respects as illustrative and not restrictive.

### EXAMPLES

The present invention will now be described with reference to specific Examples, which should not be construed as in any way limiting.

### Example One: Preparation and Characterisation of Collagen Bioinks

Materials and Methods

1.1 Preparation of Type I Collagen (Collagen I) Solutions

Collagen I powder (Bovine skin, Sigma-Aldrich) was prepared as per the manufacturer's protocol. The powder was dissolved in 0.1 M acetic acid solution (pH=2.72) under continuous stirring at a speed of 800 rpm for 16 hours. The mixture was then observed by eye to confirm that the collagen powder was fully dissolved. The collagen solution was then transferred to a 1.5 ml Eppendorf tube and stored in a fridge.

1.2 Testing the Effect of Ions on the Stability of Neutralised Collagen I Solutions

For each solution generated, 90 parts of collagen solution was neutralised with 2.7 parts of 5 M NaOH and then mixed with 7.3 parts of various salt solutions. The solutions tested included 1×Phosphate buffer saline ((PBS) consisting of NaCl, KCl, Na₂HPO₄ and KH₂PO₄), NaCl, KCl, Na₂HPO₄, KH₂PO₄, and sodium ascorbate (NaC₆H₇NO₆). The concentration of each ion tested is listed in Table 1. The solutions were left on the bench for 1 hr, 1 day, and 1 week prior to examination for precipitation formation.

TABLE 1
Concentration of ions used in testing

| | | K⁺ and | | | | | | | |
|---|---|---|---|---|---|---|---|---|---|
| Ions | PBS* | Na⁺ | Na⁺ | Ca²⁺ | K⁺ | Cl⁻ | HPO₄²⁻ | H₂PO₄⁻ | C₆H₇NO₆⁻ |
| Concentration | Na⁺: 156.9 | K⁺: 108 | 136.9 | 72.1 | 108 | 244.9 | 10 | 1.8 | 68.4 |
| (mM) | K⁺: 109.8 | Na⁺: 136.9 | | | | | | | |
| | Cl⁻: 244.9 | | | | | | | | |
| | HPO₄²⁻: 10 | | | | | | | | |
| | H₂PO₄⁻: 1.8 | | | | | | | | |
| | | K⁺: 2.7 | 68.4 | 36.0 | 1.8 | 144.2 | | | |
| | | Na⁺: 136.9 | | | | | | | |
| | | | 34.2 | 18.0 | | 136.9 | | | |
| | | | 20 | 9.0 | | 112.5 | | | |
| | | | 17.1 | | | 108 | | | |
| | | | | | | 72 | | | |
| | | | | | | 68.4 | | | |
| | | | | | | 36 | | | |
| | | | | | | 34.2 | | | |
| | | | | | | 17.1 | | | |

1.3 Generating a Collagen Bioink that is Photo-Crosslinkable

Dissolving Riboflavin in 20 Mend CaCl₂) Solution

Riboflavin at 0.1% and 0.2% (w/v) solution were made by adding 0.5 and 1 mg of riboflavin powder individually to 500 μL of 2 mg/ml CaCl₂ solution. The tubes were vortexed for 2 mins, and precipitation was checked by centrifuging the tubes in a mini centrifuge for 1 min.

Riboflavin powder (Sigma-Aldrich) was added to the freshly made neutralised collagen solutions with ions added at a final concentration of 1 mg/ml. Vortexing was necessary to fully dissolve the riboflavin powder in the collagen bioinks. The collagen bioinks with riboflavin added could be frozen at −30° C. in an Eppendorf tube wrapped in aluminium foil.

The collagen bioinks with riboflavin added were photo-crosslinked by various light sources, including 3 mw/cm² 365 nm UV, 10 mW/cm² blue light and tissue culture hood UV lamp.

1.4 Generating a Human Collagen-Based Collagen Bioink that is Photo-Crosslinkable

Collagen I powder (human skin, Sigma-Aldrich) was dissolved in 0.1 M acetic acid solution by vortexing for 10 mins. The mixture was then observed by eye to confirm that the collagen powder had fully dissolved. The collagen solution was then transferred to a 0.5 ml Eppendorf tube for further processing.

Human collagen solution (90 parts) was neutralised with 2.7 parts of 5 M NaOH and then mixed with 7.3 parts of various salt solutions. Riboflavin powder (Sigma-Aldrich) was then added to a final concentration of 1 mg/ml. The human collagen solution with riboflavin added was then photo-crosslinked by 3 mw/cm² 365 nm UV for 2 mins. Rose Bengal powder (Sigma-Aldrich) was added to the freshly made neutralised collagen solutions (the same as what used for riboflavin) at a final concentration of 1 mg/ml. The collagen ink containing Rose Bengal was further crosslinked by a white light LED, which emits 100 mw/cm² white light, for 2 mins.

1.5 Generating a Collagen-Based Collagen Bioink that is Photo-Crosslinkable Using Rose Bengal and Green Light

Rose Bengal powder (Sigma-Aldrich) was added to the freshly made neutralised collagen solutions at a final concentration of 1 mg/ml. The collagen ink containing Rose Bengal was crosslinked by a white light LED, which emits 100 mw/cm² white light, for 2 mins.

Results

1.6 Preparation of Type I Collagen (Collagen I) Solutions

It was found that up to 15 mg of the collagen I powder (bovine skin, Sigma) could be fully dissolved in 1 ml of 0.1M acetic acid without precipitation.

1.7 Testing the Effect of Ions on the Stability of Neutralised Collagen I Solutions

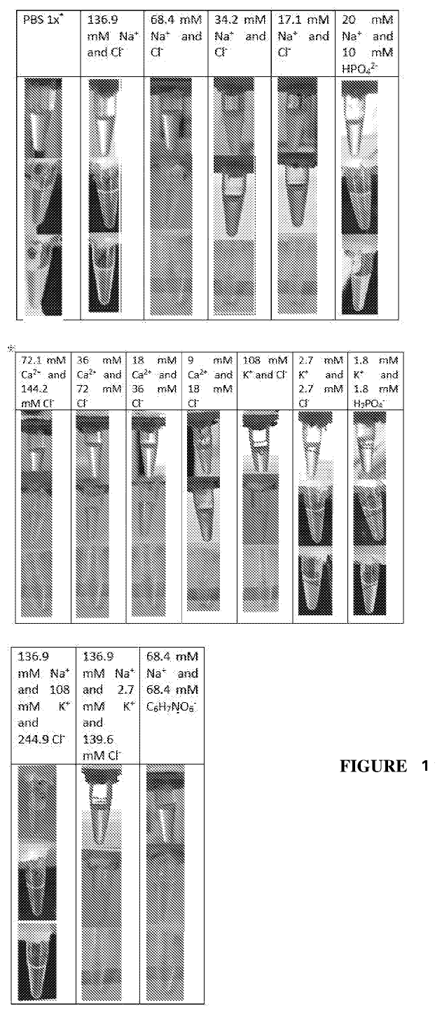

The results showed that the minimum concentration of Na to stabilize neutralised collagen solution was 136.9 mM, excluding the Na from the 135 mM NaOH. The minimum concentration of Ca2⁺ was 18 mM (FIG. 1). Combined ion results with Na⁺ at or exceeding minimal concentration also showed stable solubility of type I collagen. It was concluded that the inclusion of NaCl (at minimal concentration of 68.4 mM) or CaCl₂ (with a minimum concentration of 18 mM) was optimal to make up the soluble collagen bioinks.

1.8 Generating a Collagen Bioink that is Photo-Crosslinkable

Dissolving Riboflavin in 20 mg/ml CaCl₂) Solution

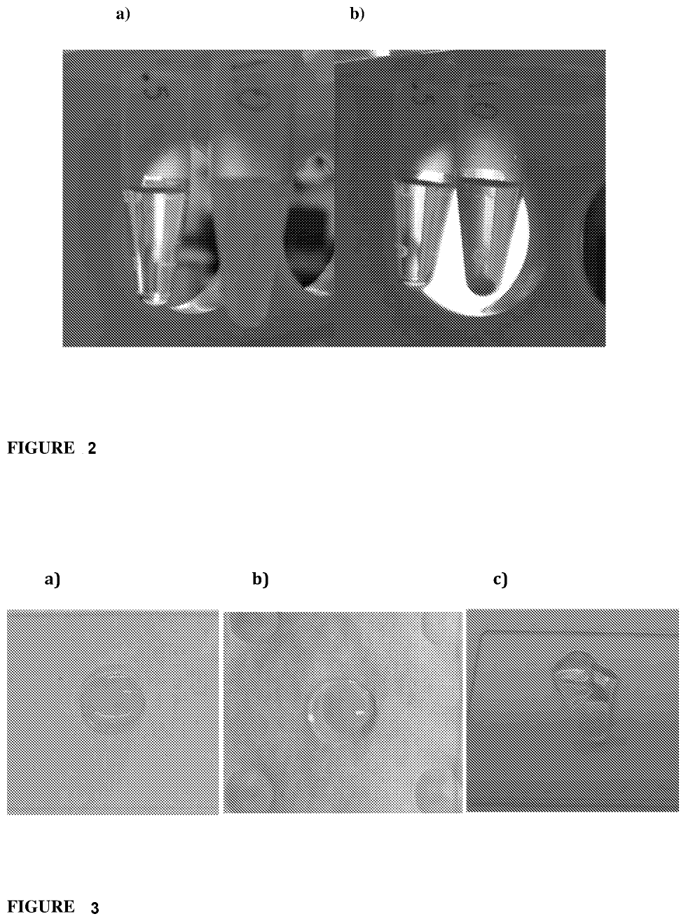

Riboflavin powder was readily dissolved in 2 mg/ml CaCl₂ (0.1% w/v, 0.5 mg in 500 uL of solution) forming a transparent yellow solution (FIG. 2a), however 1.0 mg of riboflavin did not fully dissolve (0.2% w/v) resulting a non-transparent solution (FIG. 2b).

As shown in FIG. 3, collagen bioink prepared following the methods described in sections 1.1 to 1.3 could be crosslinked by 3 mw/cm² 365 nm UV, 10 mw/cm² 470 nm blue light in 2 minutes forming a collagen gel which adhered to the glass slide. Tissue culture hood sterilization UV (254 nm UV) could crosslink the collagen bioink in 15 minutes.

1.9 Generating a Human Collagen-Based Collagen Bioink that is Photo-Crosslinkable

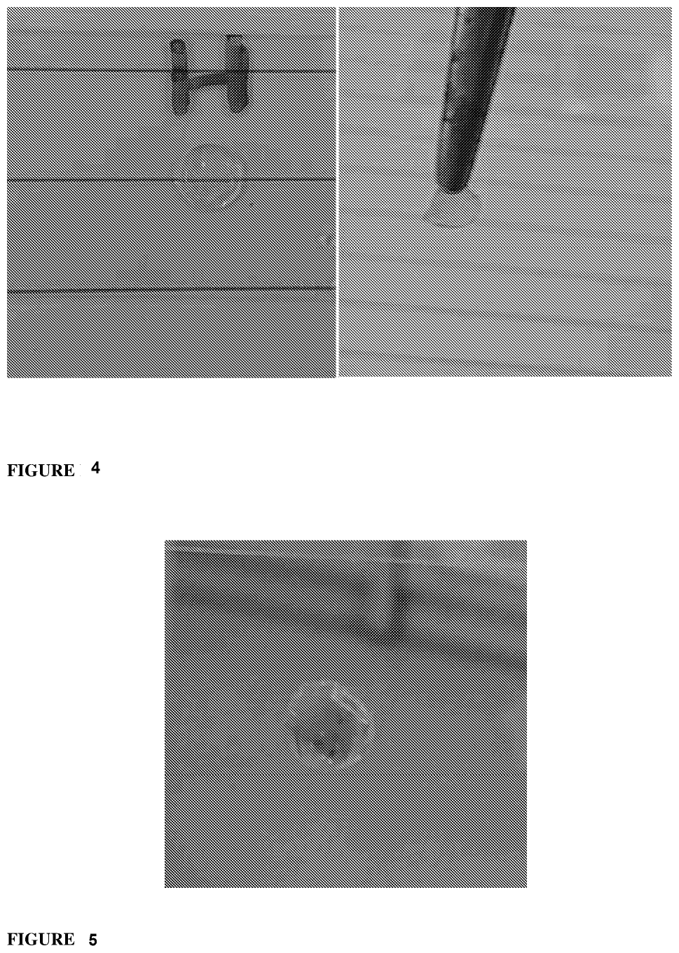

Human collagen-based collagen bioink prepared as described in section 1.4 could be crosslinked by 3 mw/cm² 365 nm UV in 2 minutes, forming a collagen gel which adhered to the glass slide (FIG. 4).

1.10 Generating a Collagen-Based Collagen Bioink that is Photo-Crosslinkable Using Rose Bengal and Green Light

The collagen type I solution with Rose Bengal added as a photo-crosslinking agent could be crosslinked by 100 mw/cm² white light in 2 minutes, forming a pink collagen gel which adhered to the glass slide (FIG. 5).

Unlike riboflavin crosslinking, the colour did not fade after rinsing as the Rose Bengal molecules were bound to the collagen type I protein. The collagen bioink crosslinkined by this method is still adhesive and could potentially be used for monitoring collagen metabolism in tissue, or for other uses requiring tracking of collagen activity.

The non-limiting Example above demonstrates the use of up to 15 mg/ml native type I collagen in the production of transparent collagen bioinks, which could be crosslinked in just two minutes to form transparent collagen gels. The Example also demonstrates the use of human collagen in the production of the bioinks.

The above Example also shows that using other ions in place of calcium is unlikely to work well. Sodium has to be present for neutralising purposes. The addition of calcium improved the storage modulus of the ink. Other ions such as potassium had to be added in an amount that well exceeded the physiological range to stabilise the collagen solution and were therefore not suitable for use in the collagen bioinks of the present invention.

### Example Two: Mechanical Testing of Collagen Bioinks

Materials and Methods

2.1 Photorheology of Collagen Bioinks

The rheological properties of both freshly made collagen bioinks and frozen collagen bioinks were examined by photorheology testing. For freshly made collagen bioink, PBS and CaCl₂ with various concentrations of collagen were tested (Table 2, samples 1-4). Riboflavin powder (Sigma-Aldrich) was mixed with the collagen solution as per the method described in section 1.3. The status of crosslinking, indicated by storage modulus, was assessed by a TA Instrument AR-G2 Rheometer coupled with an Omnicure 1000 UV lamp with a fixed 365 nm wavelength. Collagen bioink made from 12 mg/ml collagen solution, 18 mM Ca²⁺ and 1 mg/mL riboflavin was stored at −30° C. for 1 week and then thawed prior to photorefractive testing (Table 2, sample 5).

TABLE 2
Collagen bioink samples for photorheology testing

| Sample | Collagen 1 | |
|---|---|---|
| number | concentration | Buffer |
| 1 | 12 mg/ml | 1 x PBS |
| 2 | 12 mg/ml | 18 mM CaCl₂ |
| 3 | 6 mg/ml | 18 mM CaCl₂ |
| 4 | 3 mg/ml | 18 mM CaCl₂ |
| 5 | 12 mg/ml | 18 mM CaCl₂ (frozen) |

A 20 mm flat-plate (geometry gap=300 μm) geometry was used. Collagen bioink (120 μL) was loaded onto the plate. The tests were performed at room temperature. The UV strength was adjusted to 3.2 mw/cm². The collagen bioink was oscillated at 34° C., with 0.2 Hz frequency and 1% strain for 10 mins. UV was applied to the collagen bioink at the 2 min time point.

The changes in storage modulus and loss modulus over time of the collagen bioinks were recorded. The collagen bioinks after crosslinking were observed by eye.

2.2 Rotational Testing of Collagen Bioinks

For rotational testing, 60 μL of 6 mg/ml collagen bioink containing 18 mM Ca²⁺ received an increasing shear force from the geometry with a shear rate from 0.1 to 20% at 34° C. The viscosity of the collagen bioink was monitored.

2.3 Storage Modulus Measurement of Ascorbate-Incorporated Collagen Bioinks

The storage modulus of both ascorbate-incorporated collagen bioink (6 mg/ml collagen bioink containing 68.4 mM sodium ascorbate) and a control (6 mg/ml collagen bioink containing 68.4 mM NaCl) were measured using TA Instrument AR-G2 Rheometer with 15 mm diameter corn-and-plate geometry (geometry gap=55 μm). The collagen bioink was treated with 3 mw/cm² 365 nm UV for 16 seconds before measurement of the storage modulus. For storage modulus measurement, 60 μL of collagen bioink was oscillated with 0.2 Hz frequency and 1% strain for 10 mins. The storage and loss modulus of the collagen bioink was recorded

2.4 Preparation of Thin Films by Crosslinking Collagen Bioinks

A small volume of riboflavin-added collagen bioink was transferred to the surface of a glass slide. The collagen bioink on the glass slide was further expanded to cover as much area as possible without leaving a gap inside the spreading area. This was done by spreading the bioink carefully using a pipette tip. The collagen bioink was then crosslinked by 3 mw/cm² 365 nm UV curing lamp for 2 mins, followed by washing in PBS multiple times to remove riboflavin. The thickness of the crosslinked gel was measured. The flexibility and strength of crosslinked gel were observed by lifting it and immersing it in Milli-Q water using tweezers.

2.5 Spectrophotometry Measurement of Crosslinked Collagen Bioinks

The optical properties of a thin film produced by crosslinking collagen bioink composed of a 6 mg/ml collagen solution containing 18 mM Ca²⁺ and 0.1% riboflavin using the method described in section 1.3. were evaluated. The spectrophotometer was set to measure the total transmittance of the visible light range and the background was read with a round-shape 3d-printed sample holder. The collagen film was transferred carefully to the sample holder and the sample holder was then loaded into the spectrophotometer. A curve of total transmittance was obtained.

2.6 Examination of Preliminary Printing Potential Using a Line Stacking Test

A printing stage was set up on the bench whose printing area was the central area with a diameter of 2 cm of a 100 mm Petri dish. UV treatment at 3 mw/cm² was applied to the printing area constantly. Collagen bioink with a composition of 6 mg/ml collagen solution containing 18 mM Ca²⁺ and 0.1% riboflavin was slowly extruded by pipetting via a 20 μL pipette to draw a 10 mm×1 mm line. After a further UV treatment of 1 min, another line was drawn as previously described on top of the previous one. A total of 10 stacks were performed to form a 3-D structure. A simple 3-D structure was made by stacking up 10 lines drawn on top of each other. The shape and the integrity of the structure was checked by manipulation with tweezers.

Results

2.7 Photorheology of Collagen Bioinks

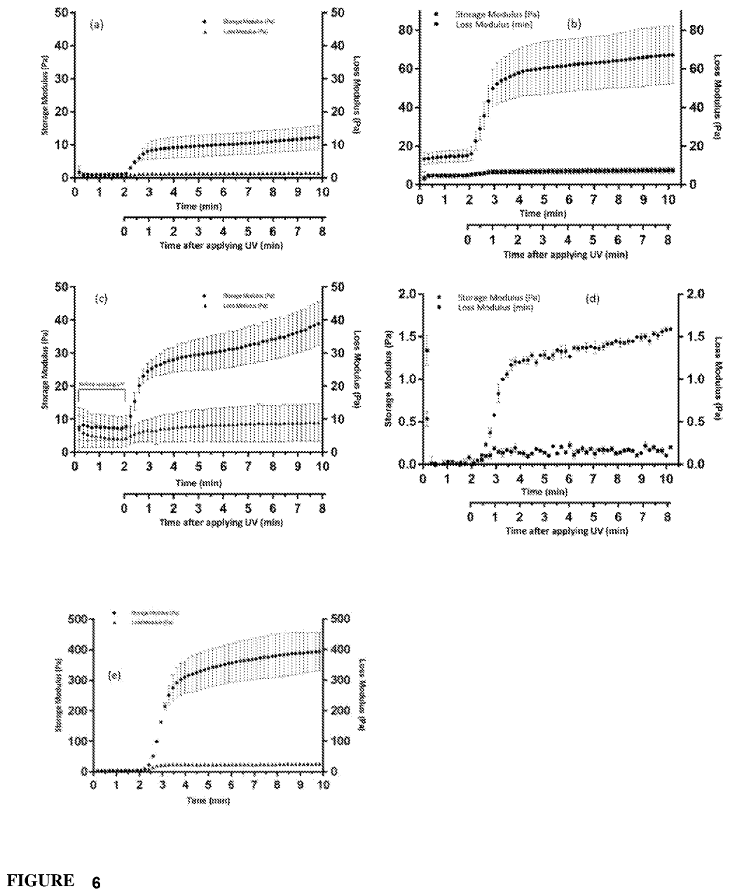

As shown in FIG. 6a, the storage modulus of collagen bioink at 12 mg/ml in 1×PBS reached 10 Pa from 1.7 Pa after 1 minute of UV irradiation, demonstrating photo-crosslinking. The resulting gel was a relatively weak gel with a storage modulus of 10 Pa (FIG. 6a). For the same concentration of collagen solution in 18 mM Ca²±, rapid crosslinking started after 1.5 mins of UV irradiation and resulted in a much stronger gel with a storage modulus of 70 Pa (FIG. 6b).

For 6 mg/ml collagen bioink containing 18 mM Ca²±, the storage modulus after crosslinking was 40 Pa, whereas 3 mg/ml collagen bioink containing 18 mM Ca²⁺ had a storage modulus of 1.5 Pa. There was no difference in the minimum UV irradiation time required for rapid crosslinking (FIGS. 6c and 6d).

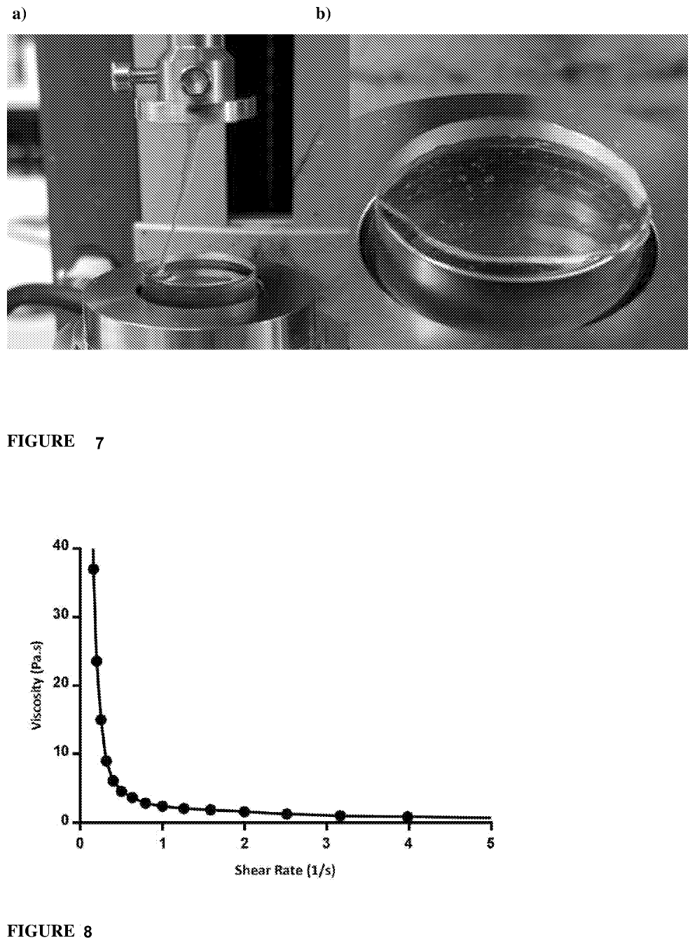

A collagen sample (6 mg/ml riboflavin-added collagen bioink containing 18 mM Ca²⁺) stored in a −30° C. freezer and thawed before testing was able to be photo-crosslinked in 1.5 mins of crosslinking time. The storage modulus of crosslinked collagen ink increased 5 times compared to a freshly-made sample with the same composition (FIGS. 6d and 6e). According to observation by eye, the collagen sample stored in the −30° C. freezer formed a more solid gel after crosslinking (FIG. 7b) in comparison to a freshly-made sample with the same composition (FIG. 7a).

2.8 Rotational Testing of Collagen Bioink

The results of rotational testing showed that the 6 mg/ml riboflavin-added collagen bioink containing 18 mM Ca²⁺ had shear thinning behaviour, with its viscosity decreasing with shear rate (FIG. 8). This property is required for extrusion 3D printing.

2.9 Storage Modulus Measurement of Ascorbate-Incorporated Collagen Bioink

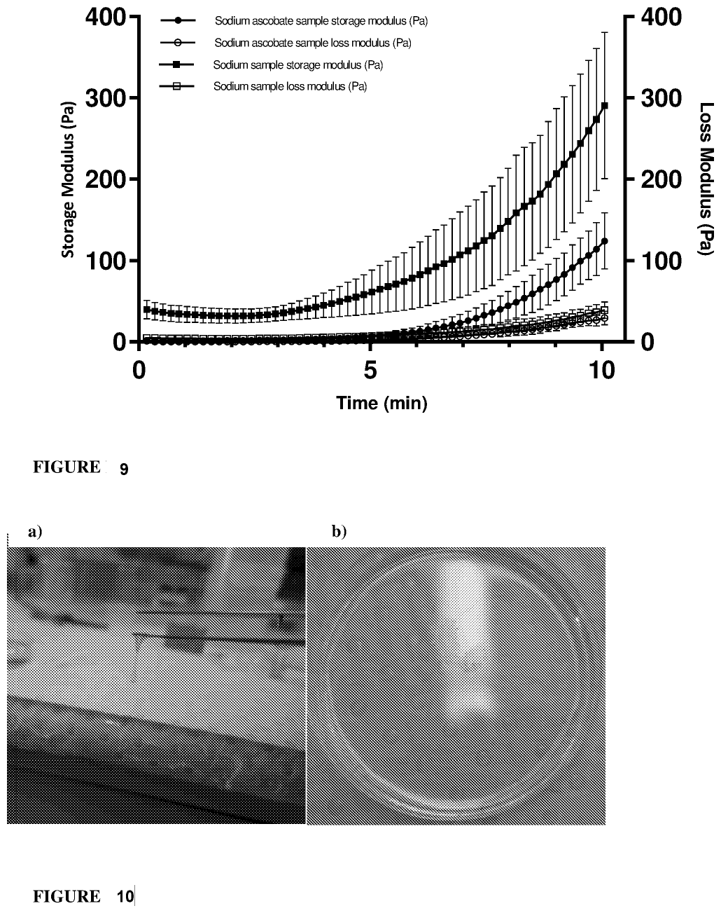

Collagen bioink incorporated with ascorbate acid showed almost half of the storage modulus at 120 kPa 10 minutes after UV treatment compared to a collagen bioink with the same collagen concentration but without ascorbate (storage modulus of 300 kPa) (FIG. 9). The strength of photo-crosslinking initiated by UV irradiation was halved with the addition of ascorbate acid.

2.10 Preparation of Collagen Gels with Various Thicknesses

The minimum thickness of generated collagen gel was measured at 100 μm. The generated collagen gel was transparent as observed by eye. It could be picked up easily with tweezers without breaking in a folded manner (FIG. 10a). Once immersed into Milli-Q water, the gel unfolded simultaneously without additional force (FIG. 10b).

2.11 Spectrophotometry Measurement of Crosslinked Collagen Bioinks

The spectrophotometry results showed that the reduction in brightness of the light passing through the crosslinked collagen bioink was less than 10% (FIG. 11). The spectrum showed that the collagen gel had a total transmittance rate of over 90% in the visible light range (FIG. 11).

2.12 Examination of Preliminary Printing Potential Using a Line Stacking Test

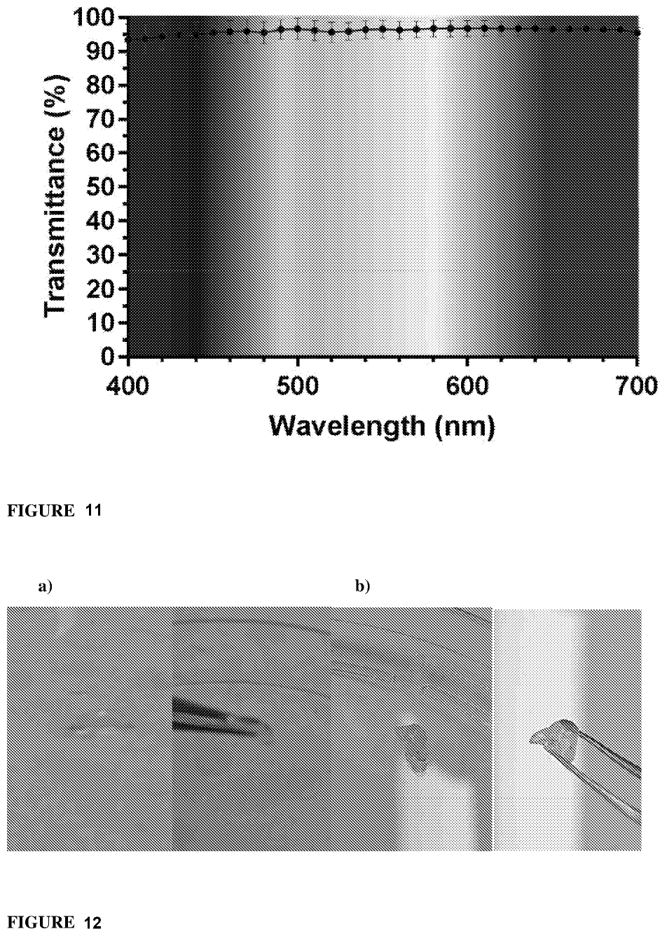

Using the method described in section 2.6, 10 lines of the 6 mg/ml collagen bioink could be stacked to form a structure and could be picked up with tweezers without separation of the layers (FIG. 12a). Collagen bioink with a collagen concentration of 12 mg/ml could also be stacked in 10 layers to form a similar structure that could also be picked up without structural damage (FIG. 12b).

The results in the non-limiting Example above show that the collagen bioinks of the present invention are capable of gelation, as the storage modulus was greater than the loss modulus. The bioinks also demonstrated shear thinning, which is an ideal property for extrusion-based 3D bioprinting.

The reduction in brightness of light passing through the crosslinked collagen bioinks was less than 10%, thus demonstrating optical clarity without impeding or substantially impeding vision arising from transmittance of light. This Example demonstrates total transmittance of the collagen gel over 90% in the visual colour range of 400-700 nm.

### Example Three: In Vitro Biocompatibility Tests of the Collagen Bioinks

Materials and Methods

3.1 Using Collagen Gel to Culture a Human Corneal Epithelial Cell Line (HCET) and Human Corneal Stromal Cells (HCSCs)

Transformed human corneal epithelial cells were cultured in HCET growth medium consisting of Dulbecco's Modified Eagle Medium/Nutrient Mixture F-12 (DMEM/F12) (Thermo Fisher Scientific), 5% (v/v) fetal bovine serum (FBS) (Sigma-Aldrich) and 10 ng/ml human epithelial growth factor (hEGF) (Thermo Fisher Scientific) in a 37° C. 5% CO₂ incubator. The cells were ready to be passaged for the experiment after reaching 85-90% confluency.

The human corneal stromal cells were obtained by culture donor tissue explants in 10% FBS cell culture medium, which was 10% FBS in DMEM/F12. The cells were ready to be passaged for the experiment after reaching 80-85% confluency.

For this experiment, collagen bioink consisted of 6 mg/ml collagen I and 1% riboflavin, and 18 mM CaCl₂ was used. Collagen bioink with a volume of 2 ml was transferred to a 35 mm Petri dish, crosslinked by tissue culture hood UV for 1 hour and rinsed with PBS multiple times to remove the yellowish colour that is caused by riboflavin.

HCET cells were seeded on top of the collagen gel at a seeding density of 5×10⁴ cells/cm². The Petri dish was kept in the 37° C. 5% CO₂ incubator. HCSCs were also seeded on top of the collagen gel at a seeding density of 5×10⁴ cells/cm² and kept in the 37° C. 5% CO₂ incubator. The cell culture medium was changed every two days. Cell growth was observed by Olympus IX71 inverted microscope.

When the cells reached confluency on the surface of the collagen gel, a part of the collagen gel was removed using a 4 mm diameter trephine. The harvested collagen gel piece was fixed and cryo-sectioned to 20 μm sections. DAPI was used to stain the nuclei of the cells in the sections.

3.2 Cell Delivery Experiment Using HCET

Collagen bioink consisting of 6 mg/ml collagen I, 1% riboflavin, and 18 mM CaCl₂) was used for this experiment. The collagen bioink was mixed with HCET cells in their growth medium to obtain a density of 1×10⁶ HCET cells/ml. The collagen bioink with cells was then slowly pipetted using 200 μL pipette with a 200 μL pipette tip to draw 3 lines in a 35 mm Petri dish. The three lines of the collagen bionics then received 2 minutes of 3 mw/cm² 365 nm UV treatment. 2 ml of cell culture medium was added to the Petri dish after UV treatment. The Petri dish was kept in the 37° C. incubator with 5% CO₂. The cell culture medium was changed every two days. The Petri dish was observed using an Olympus IX71 inverted microscope each day.

3.3 Cell Delivery Experiment Using hCSCs

Collagen bioink consisting of 6 mg/ml collagen I, 1% riboflavin, and 18 mM CaCl₂) was used for this experiment. The collagen bioink was mixed with human cornea stromal cells (hCSCs) in their growth medium to obtain a density of 5×10⁴ cells/ml. The collagen bioink with cells was then slowly pipetted using a 200 μL pipette with a 200 μL pipette tip to draw a line in a 35 mm Petri dish. The line of the collagen bioink then received 1 minute of 3 mw/cm² 365 nm UV treatment. 2 ml of cell culture medium was added to the Petri dish after UV treatment. The Petri dish was kept in a 37° C. incubator with 5% CO₂. The cell culture medium was changed every two days. The Petri dish was observed using an Olympus IX71 inverted microscope each day.

3.4 Cell Proliferation Test Using Bromodeoxyuridine/5-Bromo-2′-Deoxyuridine

Bromodeoxyuridine/5-bromo-2′-deoxyuridine (BrdU) is a chemical that can be incorporated into DNA during its synthesis, and therefore can be used to measure cell proliferation. A higher BrdU reading indicates a higher amount of DNA synthesized, which means more cell proliferation.

The BrdU Cell Proliferation ELISA Kit (chemiluminescent) from Abcam was used. Based on the manufacturer's instructions, HCET cells (1000 cells/well) or human corneal stromal cells (hCSCs) (3000 cells/well) were seeded into a 96 well plate. Cells were tested in two conditions: cells seeded on top of collagen gel with BrdU stain (experimental group 1), cells seeded on top of collagen gel without BrdU stain (BrdU background 1), cells seeded without collagen gel but containing BrdU stain (experimental group 2) and cells seeded without collagen gel with BrdU stain (BrdU background 2) (Table 3).

TABLE 3
Experimental groups for the BrdU Cell Proliferation ELISA
Kit for HCET and human corneal stromal cells

| | |
|---|---|
| Experimental group 1 | Cells on top of collagen gel (BrdU stain) |
| BrdU background 1 | Cells on top of collagen gel (no BrdU |
| | stain) |
| Experimental group 2 | Cells without collagen gel (BrdU stain) |
| BrdU background 2 | Cells on top of collagen gel (no BrdU |
| | stain) |

The collagen bioink used in this experiment to form the collagen gel consisted of 6 mg/ml collagen, 1% riboflavin and 18 mM Ca²⁺.

HCET cells were cultured in DMEM/F12 containing 5% FBS. BrdU was added to the designed wells 2 hours before the end of culturing. Human corneal stromal cells were cultured in serum-free keratocytes growth medium consisting of Dulbecco's Modified Eagle Medium/Nutrient Mixture F-12 (DMEM/F12) (Thermo Fisher Scientific), 1% (v/v) Insulin-Transferrin-Selenium-Ethanolamine (ITS-X) (Thermo Fisher Scientific), 10 ng/ml basic fibroblast growth factor (FGF-basic) (Thermo Fisher Scientific) and 1 mM ascorbate acid. BrdU was added to the designated wells 4 hours before the end of culturing.

Results

3.5 Using Collagen Gel to Culture a Human Corneal Epithelial Cell Line (HCET) and Human Corneal Stromal Cells (HCSCs)

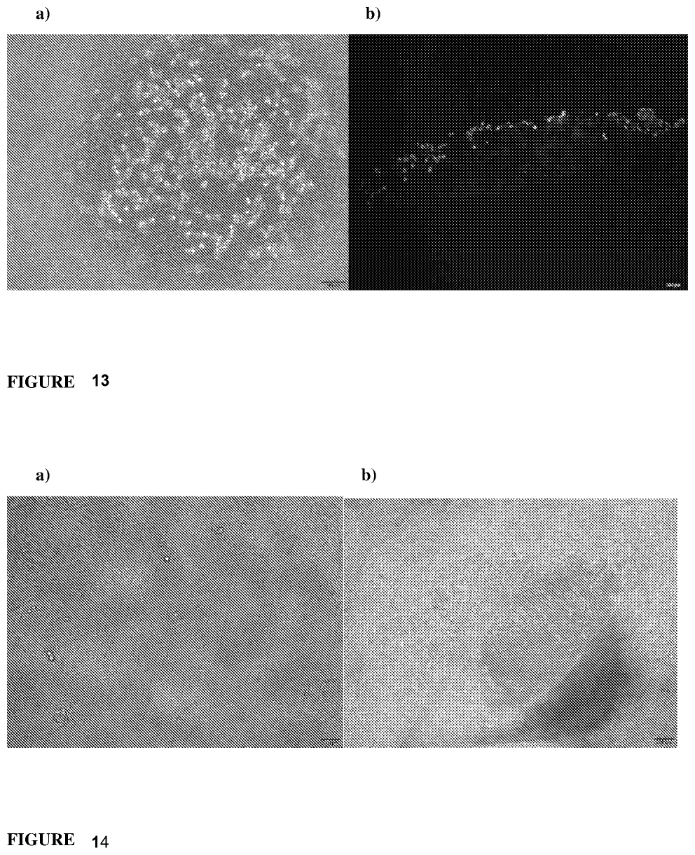

Phase contrast microscopy showed that HCET cells could reach confluence within 7 days of cell culture on the surface of the collagen gel (FIG. 13a). DAPI staining (which stains the nucleus of the cells) of a cross-section of the cell-seeded collagen gel showed that some of the cells migrated into the collagen gel body during cell culture (FIG. 13b).

HCSCs also reached confluence within 7 days (FIG. 14a). Degradation of the collagen gel could be observed when the cells reached confluence (FIG. 14b).

3.6 Cell Delivery Experiment Using HCET

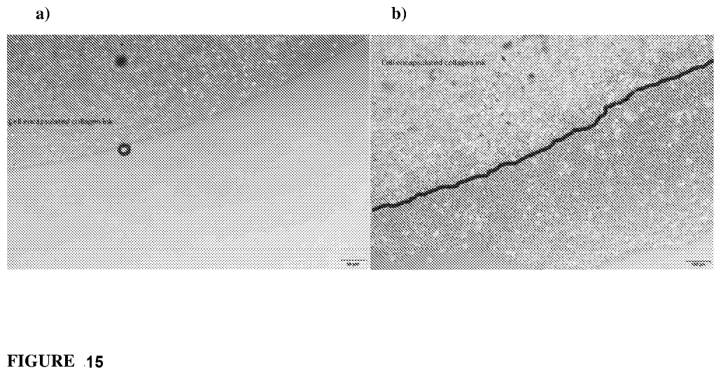

FIG. 15a shows the cells encapsulated in the crosslinked collagen on day 1. Cell migration and proliferation could be observed on day 5 while the structure of the crosslinked collagen bioink was still intact after cell migration (FIG. 15b).

3.7 Cell Delivery Experiment Using hCSCs

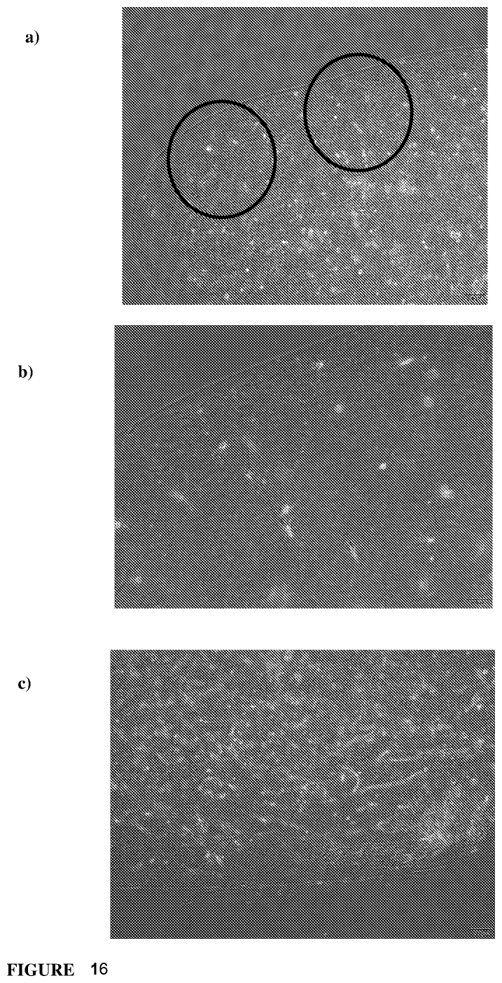

The images in FIG. 16 show that cell migration and proliferation inside the collagen gel body were observed on day 3 and cells started to migrate out of the collagen gel on day 7. No degradation of the collagen gel was observed during the process.

3.8 Cell Proliferation Test Using Bromodeoxyuridine/5-Bromo-2′-Deoxyuridine

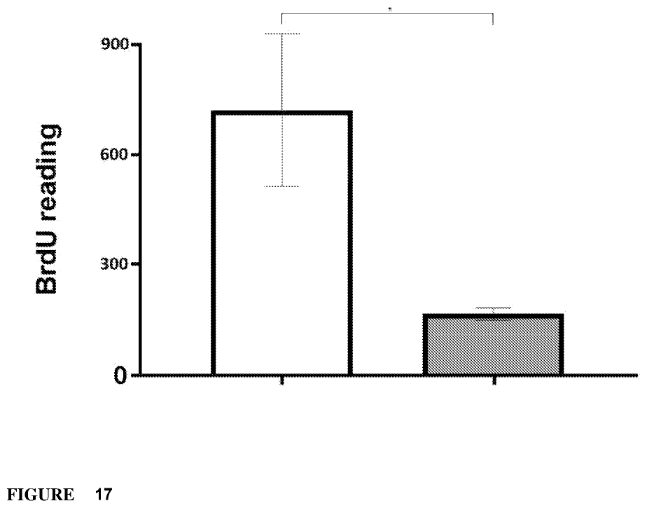

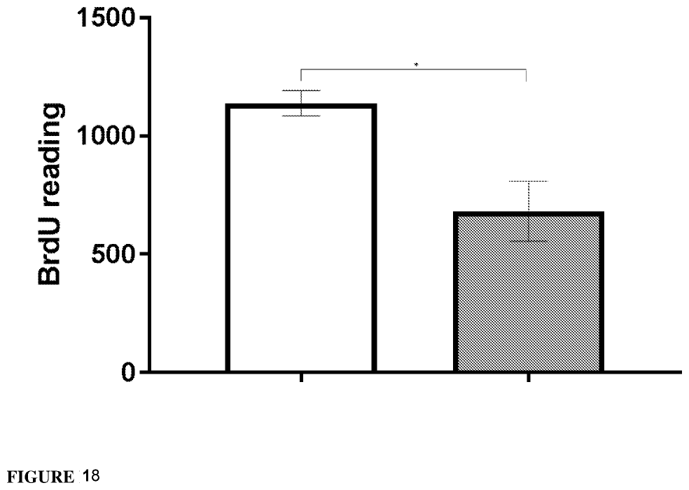

BrdU readings of HCET cells on top of the collagen gel were significantly higher (p=0.04) than those for the cells on the tissue culture plate following 2 hours incubation with BrdU (FIG. 17). Human corneal stromal cells on top of the collagen gel also showed a significantly higher BrdU reading (p=0.01) than the cells on the tissue culture plate after 4 hours incubation with BrdU (FIG. 18).

The results in this Example show that the collagen bioinks of the present invention have high cell compatibility. The bioinks supported cell proliferation and migration of human corneal epithelial cells and human corneal stromal cells when seeded on top of the collagen gels. The results also demonstrated that UV photo-crosslinking did not affect the survival of encapsulated cells. The results show that collagen bioinks of the present invention can be used for cell delivery.

### Example Four: Generation of Different Versions of the Collagen Bioinks

Materials and Methods

4.1 Preparation of Collagen Bioinks Incorporated with Collagen IV

Collagen IV powder was dissolved in acidic 6 mg/ml collagen solution to a concentration of 0.2 mg/ml. The collagen IV-incorporated collagen solution was further neutralised prior to the addition of 18 mM calcium ions and 0.1% riboflavin to produce a photo-crosslinkable collagen IV-incorporated collagen ink.

4.1.1 Corneal Endothelial Cell Line Cultured on the Surface of the Collagen Gel

A human corneal endothelial cell line (HCEC-B4G12) was cultured on top of the crosslinked collagen bioink incorporated with collagen IV with a cell density of 5×10⁴ cells per cm². The culture medium was 5% Fetal Calf Serum (FCS), 20 μg/ml ascorbic acid, 20 μg/ml insulin, and 10 ng/ml FGF-basic in 1:1 mixture of Nutrient Mixture Ham's F12 and Medium 199 (F99 medium). The cells were cultured in a 37° C. 5% CO₂ incubator and the cell culture medium was changed every two days.

4.2 Preparation of Collagen Bioink Incorporated with FBS

Sterilised collagen bioink consisting of 12 mg/ml collagen I and 18 mM CaCl₂) was used. Nine parts of collagen ink were mixed with one part of FBS, yielding a collagen ink containing 10% FBS. The collagen ink containing 10% FBS was then photo-crosslinked to form a collagen gel.

4.2.1 Cell Proliferation Test

The BrdU Cell Proliferation assay was performed following the method described in section 3.4. Human corneal fibroblasts were cultured in DMEM/F12 medium with a seeding density of 2000 cells per well. BrdU was added to the designated wells 20 hours before the end of culturing.

Results

4.3 Collagen Bioink Incorporated with Collagen IV

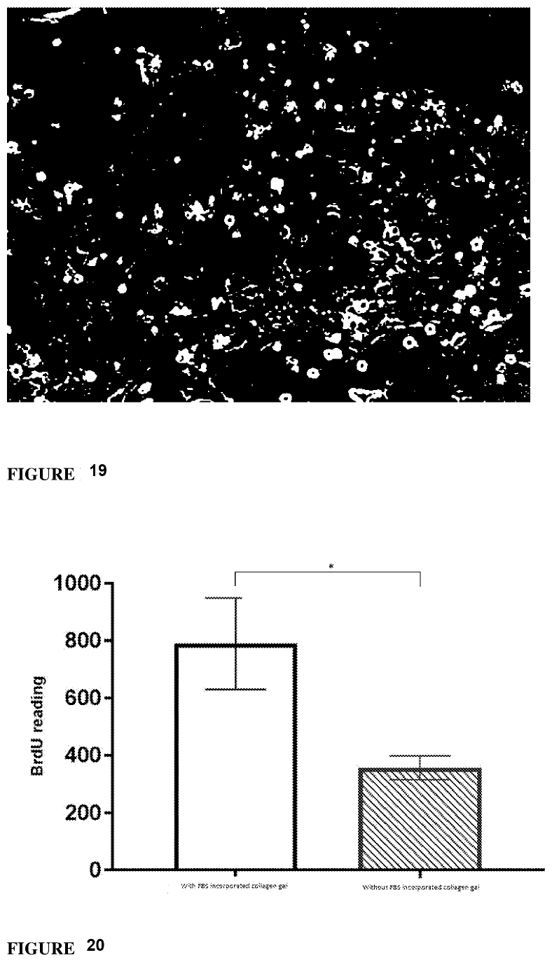

A human corneal endothelial cell line (HCEC-B4G12) cultured on top of the crosslinked collagen IV-incorporated 6 mg/ml collagen ink reached confluence in 7 days (FIG. 19).

′4.4 Collagen Bioink Incorporated with FBS

Human corneal stromal cells cultured on the surface of an FBS-incorporated collagen gel showed higher BrdU readings (FIG. 20), indicating higher cell proliferation, than the HCSCs seeded without FBS-incorporated collagen gel.

The results in this Example show that a photo-crosslinked collagen gel can be readily made with collagen type I bioink mixed with collagen type IV. As collagen type IV is a major component of Descemet's membrane, which is the layer that corneal endothelial cells adhere to, the combination can provide further benefit to corneal endothelial cell culture.

### Example Five: Ex Vivo Filling/Sealing of a Porcine Cornea

Materials and Methods

5.1 Filling Corneal Stroma with Collagen Bioink

Two types of holes were created in the corneas-stromal holes and perforations. A porcine cornea was mounted onto an anterior chamber and a portion of anterior cornea up to 0.5 mm deep was removed using a 2- or 4-mm diameter trephine resulting in non-penetrating damage to the stroma. Collagen inks with 6 and 12 mg/ml collagen, 1% riboflavin and 18 mM Ca²⁺ as prepared in Section 6.1 were extruded under 3 mw/cm² 365 nm UV or 10 mw/cm² blue light curing to fill the hole. After the hole was filled, an additional 2 min UV irradiation time was applied. The adhesiveness of collagen ink was examined by rinsing the cornea under running water for 30 seconds followed by drying with kimwipes.

5.2 Sealing Perforations with Collagen Bioinks

An IOP simulating system was set up to measure the burst pressure of the collagen ink sealed cornea. The IOP simulating system included a height-adjustable syringe connected to an anterior chamber using a medical polyethylene tube. Pressure was created by adding water to the syringe and quantified by converting the height of the water to the height of mercury (mmHg). Two pressure points of 22 mmHg and 50 mmHg were tested for the normal human IOP and extremely high IOP.

A perforation was created using a 0.8 mm diameter needle, 1.5 mm diameter needle or a 2 mm diameter trephine to penetrate a porcine cornea that was secured on an anterior chamber. Water was continuously added into the syringe to maintain the pressure. 6 mg/ml and 12 mg/ml collagen bioink were then applied to seal the perforation under UV irradiation or blue light curing as described in section 5.1 above. Milli-Q water was then pumped into the anterior chamber to restore the pressure. The condition of the crosslinked collagen bioink and leaking was examined by eye.

Results

5.3 Filling Corneal Stroma with Collagen Bioink

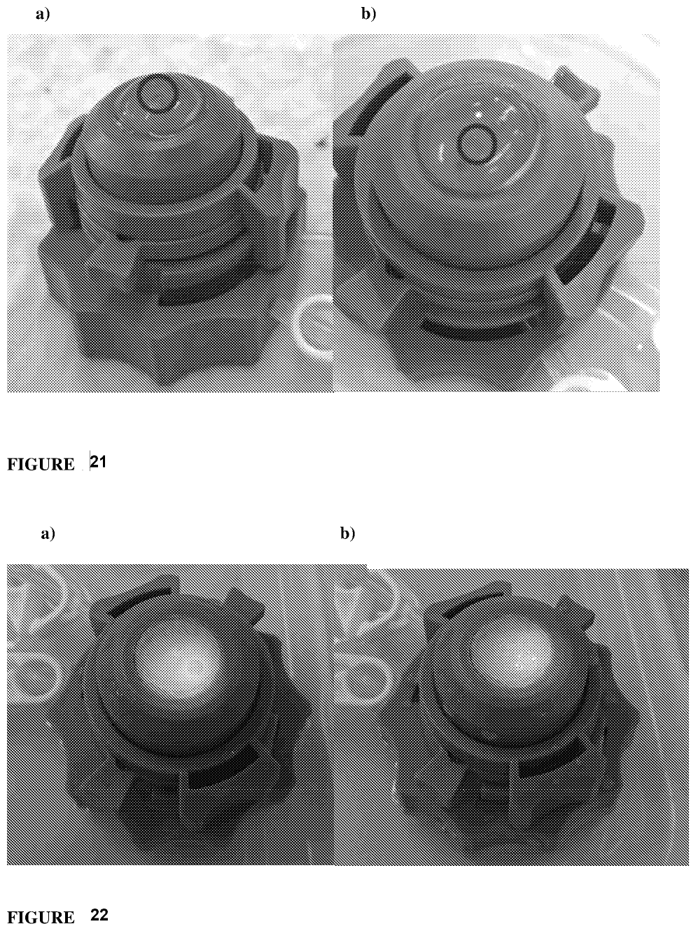

A non-penetrating hole created in a pig cornea is shown in FIG. 21a (circled). The stage of the gap and the 6 mg/ml crosslinking collagen bioink applied to the hole after 30s of rinsing with running water are shown in FIG. 21b (circled). 12 mg/ml collagen ink yielded the same result (FIGS. 22a and 22b). The collagen bioink could also fill the larger hole (4 mm diameter) under blue light curing (FIG. 23).

5.4 Sealing Perforations with Collagen Ink

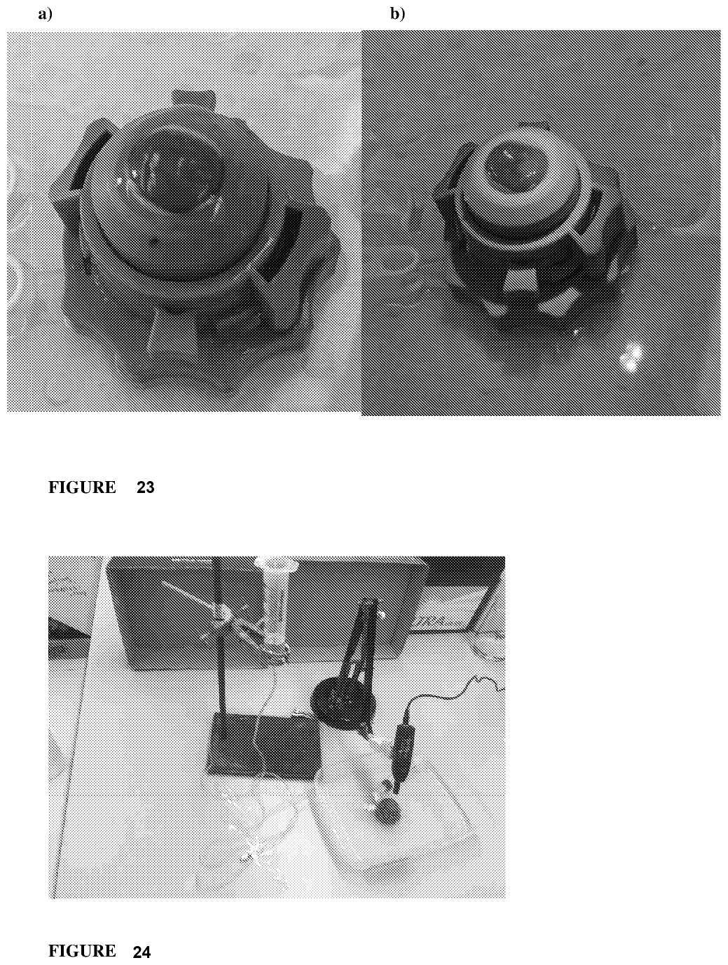

The IOP simulating system used in this Example is shown in FIG. 24. Collagen bioink with a collagen concentration of 6 mg/ml was able to seal the 0.8 mm diameter perforation and stop the perforation leaking at 22 mmHg and 50 mm mmHg IOP (FIG. 25a). Collagen bioink with a collagen concentration of 12 mg/ml failed to adhere to the perforated area at 22 mmHg IOP (FIG. 25b).

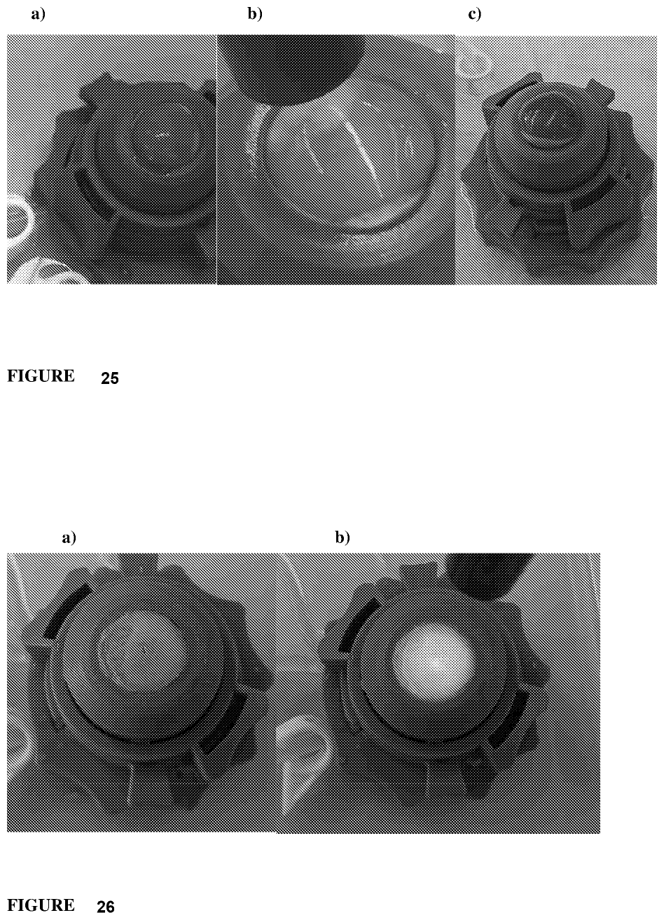

12 mg/ml collagen ink could seal a 1.5 mm diameter perforation and stop the leaking up to 50 mmHg IOP, while 6 mg/ml collagen ink fail to seal the perforation (FIG. 26a, FIG. 26b).

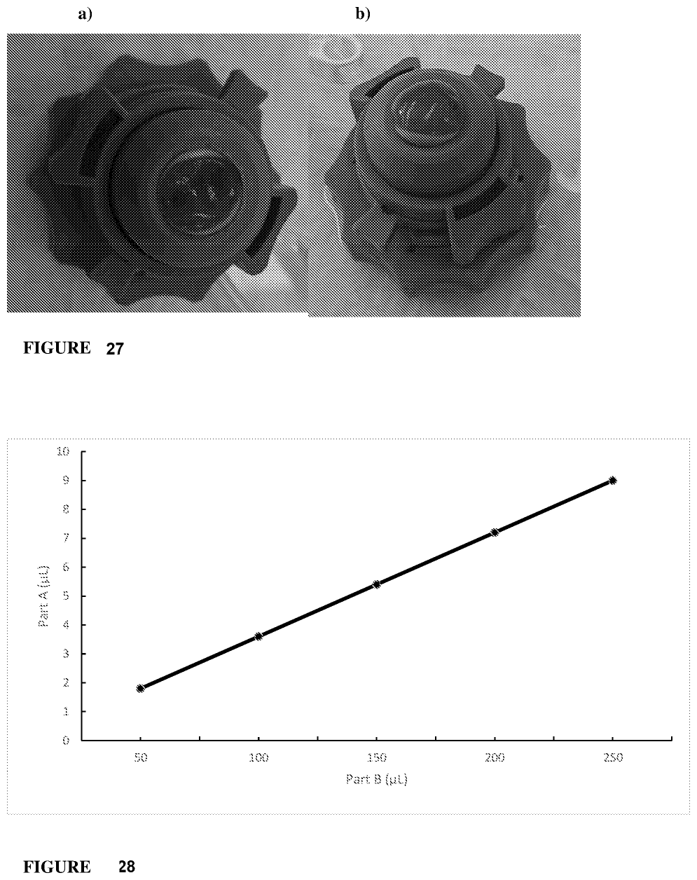

12 mg/ml collagen ink could seal a 2 mm diameter perforation at 22 mm Hg but failed to prevent leaking at higher IOP (FIG. 27a, FIG. 27b).

The results in this Example show that 6 mg/ml of collagen bioink can be directly applied to cornea with sufficient adhesiveness, and therefore can be used as a sealant for corneal tissue. For sealing corneal tissue, collagen bioink at 6 mg/ml collagen concentration with 18 mM calcium ions appears to be the optimal composition. The results in Sections 5.3 and 5.4 show that collagen ink at 12 mg/m can seal a much larger perforation provided that the perforation is not leaking. For example, after using other surgical tools or membrane/film to block the leaking, 12 mg/ml collagen ink will be able to seal a perforation that is greater than 4 mm diameter.

### Example Six: A Refined Method of Generating Crosslinkable Collagen Ink

Materials and Methods

6.1 Preparation of Parts A and B

Riboflavin powder (Sigma-Aldrich) was added to 5 M NaOH at a concentration of 30 mg/ml, forming part A of the collagen bioink. Ions were added to 12 mg/ml acetic acid collagen solution at the concentrations listed in Table 4, forming part B of the collagen bioink. Following their preparation, parts A and B were mixed in a 9:250 ratio in an Eppendorf tube to obtain neutralized collagen bioink containing riboflavin, which was ready to be crosslinked.

6.2 Storage of the Collagen Bioink

Acidic collagen solution/neutralized collagen solution containing riboflavin (collagen bioink) was prepared as mentioned in Section 6.1 above and stored in a fridge (4° C.) and freezer (−20° C.). The acidic collagen solution was stored in 1.5 ml Eppendorf tubes, while the collagen bioink was stored in aluminium foil-wrapped Eppendorf tubes. Both solutions were checked after 1 week/1 month/half year and one year by neutralizing and crosslinking under 365 nm 3 mw/cm² UV or directly crosslinking under UV.

6.3 Preparation of Collagen Membrane by Crosslinking Collagen Ink

A small volume of riboflavin-added collagen bioink was transferred to the surface of a paraffin-wrapped glass slide. Two size 0 coverslips with a thickness of 0.1 mm per slide were added to both sides of the glass slide, with at least 1 cm distance to the collagen drop. Then a 1 mm thick paraffin-wrapped Polypropylene plate was placed on top of the glass slide, which flattened the collagen drop to 0.1 mm thick. The collagen ink was then crosslinked by a 3 mw/cm² UV curing lamp or a 10 mw/cm² blue light curing lamp for 3 mins. The membrane was removed from the glass slide and washed in PBS after crosslinking. The flexibility and strength of the membrane were observed by a rolling and self-expanding test.

6.4 3D Printing Testing

The developed collagen ink was subjected to a 3D printing test. A 3D printer (TRICEP, University of Wollongong) was used. The collagen ink was loaded in an aluminium foil-wrapped 5 mm diameter syringe with a 25-gauge printing tip. 10 mw/cm² 470 nm blue light was used to assist printing and crosslinking the collagen bioink. For a mesh printing test, the extrusion rate was set to 0.5 mm/min and the printing speed was set to 150 mm/min.

6.5 Cell Printing Test to Print a Cornea-Shaped Structure Loaded with Cells

The printer used in Section 6.4 above was set up in a sterile bio-printing hood sterilized by 20 mins of UV. The collagen ink was prepared by the method described in Section 6.1 above with part B also receiving 20 mins of UV sterilization. After mixing part A and part B, the collagen bioink was further mixed with 10% FBS DMEM/F12 cell culture medium containing a cell number of 2 million cells per millilitre at a 9:1 ratio and then loaded in the same syringe used in Section 6.4 above in the sterile hood. The printed structure was cultured in the tissue culture medium. Calcein-AM staining which stains live cells was performed 2 weeks after printing to check cell viability.

Results

6.6 Titration of Part a and Part B of the Crosslinkable Collagen Bioink.

50, 100, 150, 200, and 250 μL of collagen ink part B (acidic collagen ink with calcium ions) were mixed with specific volumes of collagen ink part A to make the bioink reach neutral pH and be crosslinkable. The amount of part A needed for each volume of part B is showed in Table 4. The relationship of the volumes of part A and B was linear (FIG. 28), showing that part A and part B had a fixed ratio of 0.036 and can be prepared easily by following the ratio.

TABLE 4
The amount of part A needed for each volume of part B

| Part A | Part B |
|---|---|
| 1.8 μL | 50 μL |
| 3.6 μL | 100 μL |
| 5.4 μL | 150 μL |
| 7.2 μL | 200 μL |
| 9.0 μL | 250 μL |

6.6 Storage of the Collagen Bioink

Results of the storage tests are provided in Table 5.

TABLE 5
Properties of the collagen ink stored under different conditions

| | Acidic collagen solution | | Collagen ink | |
|---|---|---|---|---|
| | | Freezer | | Freezer |
| | Fridge (4° C.) | (−20° C.) | Fridge (4° C.) | (−20° C.) |
| 1 week | crosslinkable | crosslinkable | Self- | crosslinkable |
| | | | crosslinked | |
| 1 month | crosslinkable | crosslinkable | — | crosslinkable |
| 6 months | crosslinkable | crosslinkable | — | crosslinkable |
| 12 months | crosslinkable | crosslinkable | — | crosslinkable |

6.8 Preparation of Collagen Membranes with Various Thicknesses

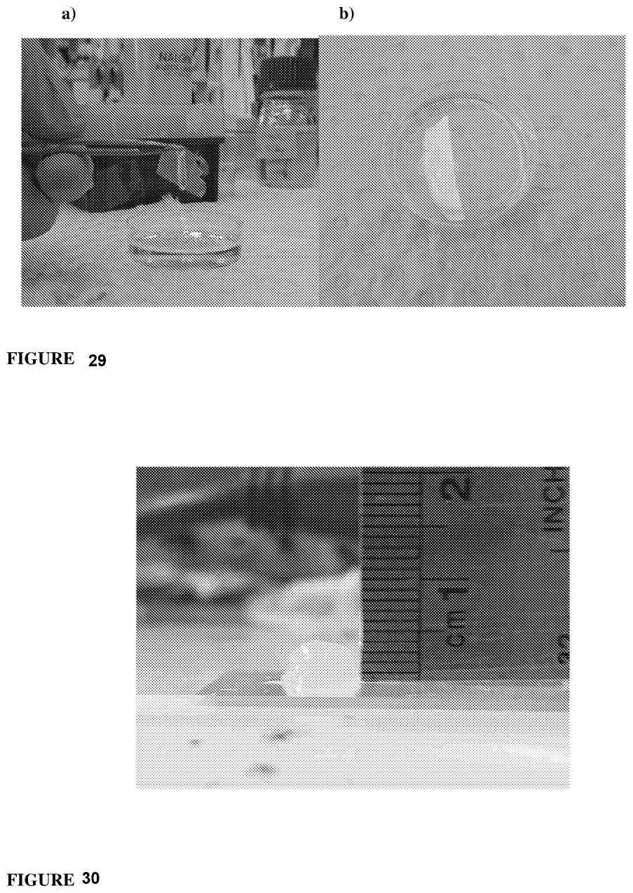

The collagen membrane could be generated by blue light curing. The minimum thickness of generated collagen gel was measured at 100 μm. The generated collagen gel was transparent judged by observation by eye. The gel could be picked up easily using tweezers without breaking in a folded manner (FIG. 29a). Once immersed into Milli-Q water, the gel unfolded simultaneously without additional force (FIG. 29b). A crosslinked collagen structure generated by this method can be as thick as 4 mm (FIG. 30).

6.9 Printing Test Using 3D Extrusion Printer

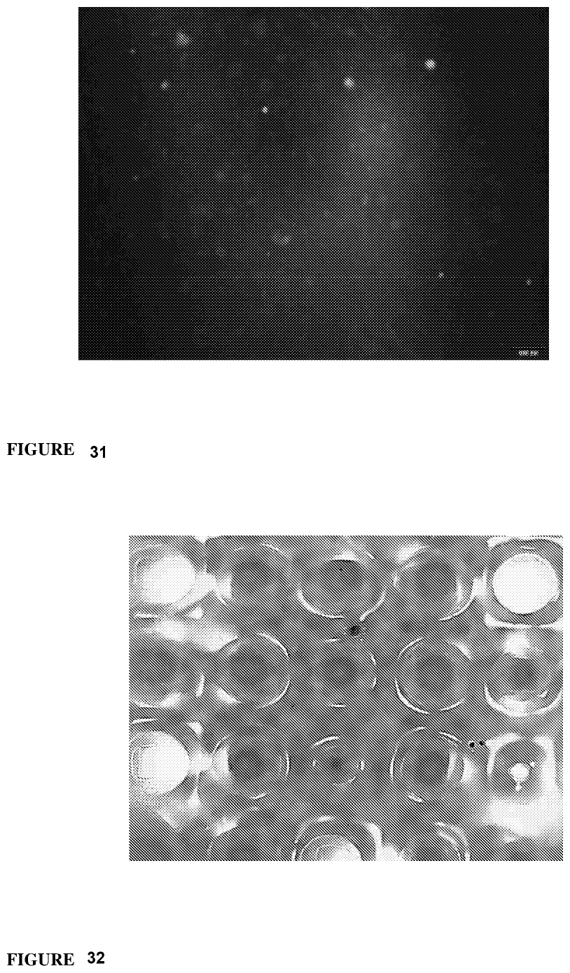

A double layer 10 cm×10 cm mesh was printed using the printer with the method described in 6.4 above (FIG. 31). The lines of the mesh did not join after printing, which shows the collagen ink is suitable for 3-D printing and compatible with an extrusion 3-D printer with a UV/blue light curing attachment.

6.10 Cell Printing Test to Print a Cornea-Shaped Structure Loaded with Cells

A cell-loaded structure was printed using the method described in Section 6.5. The printed structure was transferred to the cell culture medium after printing. Calcein-AM staining showed that the cells were still viable after 2 weeks (FIG. 32).

1. A composition comprising:
3-15 mg/ml type I collagen;
0.135-0.5 M sodium ions and/or 0.008-0.4 M calcium ions; and
one or more crosslinking agents.

2. The composition of claim 1, wherein the composition comprises 0.01-0.5% (w/v) riboflavin or 0.01-0.5% (w/v) Rose Bengal.

3. The composition of claim 1 or claim 2, wherein the one or more crosslinking agents are capable of activation by light.

4. The composition of claim 3, wherein the light is UV light, blue light, green light or white light.

5. The composition of claim 1, wherein the composition comprises fibrinogen and/or thrombin.

6. The composition of claim 5, wherein the composition comprises 1.6-6 mg/ml fibrinogen.

7. The composition of claim 5 or claim 6, wherein the composition comprises 1-5 U/mL thrombin.

8. The composition of any one of claims 1 to 7, further comprising any one or more of: a culture medium, growth factors, hormones, matrix proteins, glycoproteins, vitamins, ions other than sodium ions or calcium ions, ion sources, fibronectin, amino acids, antibiotics, anaesthetics, factor XIII, Fetal Bovine Serum (FBS), human serum, platelet lysate, human platelet lysate.

9. The composition of claim 8, wherein the composition comprises a culture medium comprising the ions and amino acids.

10. The composition of claim 8 or claim 9, wherein:
(i) the growth factors comprise human epidermal growth factor (hEGF) and/or fibroblast growth factor (FGF); and/or
(ii) the vitamins comprise ascorbate (vitamin C); and/or
(iii) the matrix proteins comprise collagen IV; and/or
(iv) the hormones comprise insulin; and/or
(v) the glycoproteins comprise transferrin.

11. The composition of any one of claims 1 to 10, wherein the ions are components of an ionic salt included in the composition.

12. The composition of any one of claims 1 to 11, wherein the composition further comprises mammalian cells.

13. The composition of claim 12, wherein the mammalian cells comprise or consist of human cells.

14. The composition of any one of claims 1 to 13, wherein the type I collagen is neutralised.

15. The composition of any one of claims 1 to 14, wherein the composition comprises:
(i) 3-15 mg/ml type I collagen, 0.135-0.2 M sodium ions, and 0.01-0.05 M calcium ions; or
(ii) 4-12 mg/ml type I collagen, 0.135-0.16 M sodium ions, and 0.015-0.03 M calcium ions; or
(iii) 5-11 mg/ml type I collagen, 0.135-0.14 M sodium ions, and 0.018-0.02 M calcium ions.

16. The composition of any one of claims 1 to 15, wherein the composition comprises:
(i) less than 15 mg/ml type I collagen;
(ii) more than 0.135 M sodium ions; and
(iii) more than 0.018 M calcium ions.

17. A method of preparing a composition, the method comprising:
(i) providing a solution comprising:
3-15 mg/ml type I collagen;
one or more crosslinking agents; and
0.135-5 M sodium ions; and/or 0.008-0.4 M calcium ions;
(ii) applying the solution to a surface; and
(iii) applying light capable of activating the one or more crosslinking agents to the solution.

18. The method of claim 17, wherein applying the solution to a surface forms a layer, and wherein steps (ii) and (iii) are repeated a plurality of times, wherein each layer is applied on top of the preceding layer.

19. The method of claim 17 or claim 18, wherein the one or more crosslinking agents comprise 0.01-0.5% (w/v) riboflavin or 0.01-0.5% (w/v) Rose Bengal, and wherein the light comprises UV light, blue light, green light or white light.

20. The method of any one of claims 17 to 19, wherein:
(a) the one or more crosslinking agents are combined with a base to form part A;
(b) the type I collagen is combined with the sodium ions and/or calcium ions to form part B; and
(c) parts A and B are mixed to form a solution prior to step (iii).

21. A method of preparing a composition, the method comprising:
(i) providing a solution comprising:
3-15 mg/ml type I collagen;
one or more crosslinking agents; and
0.135-5 M sodium ions; and/or 0.008-0.4 M calcium ions; and
(ii) applying the solution to a surface, wherein the solution is divided into at least two components prior to applying to the surface.

22. The method of claim 21, further comprising the steps of:
(iii) adding fibrinogen to at least one component to form formulation (a);
(iv) adding thrombin to at least one component to form formulation (b); and
(v) combining formulations (a) and (b) to form a gel.

23. The method of claim 22, wherein:
1.6-6 mg/ml fibrinogen is added to at least one component to form formulation (a) in step (iii); and/or
1.5 U/mL thrombin is added to at least one component to form formulation (b) in step (iv).

24. The method of any one of claims 17 to 23, wherein the solution further comprises any one or more of: a culture medium, growth factors, hormones, matrix proteins, glycoproteins, vitamins, ions other than sodium ions or calcium ions, ion sources, fibronectin, amino acids, antibiotics, anaesthetics, factor XIII, Fetal Bovine Serum (FBS), human serum, platelet lysate, human platelet lysate.

25. The method of claim 24, wherein the solution comprises a culture medium comprising the ions and amino acids.

26. The method of claim 24 or claim 25, wherein:
(i) the growth factors comprise human epidermal growth factor (hEGF) and/or fibroblast growth factor (FGF); and/or
(ii) the vitamins comprise ascorbate (vitamin C); and/or
(iii) the matrix proteins comprise collagen IV; and/or
(iv) the hormones comprise insulin; and/or
(v) the glycoproteins comprise transferrin.

27. The method of any one of claims 17 to 26, wherein the ions are components of an ionic salt included in the mixture.

28. The method of any one of claims 17 to 27, wherein the solution further comprises mammalian cells.

29. The method of claim 28, wherein the mammalian cells comprise or consist of human cells.

30. The method of any one of claims 17 to 29, wherein the type I collagen is neutralised.

31. A composition obtained or obtainable by the method of any one of claims 17 to 30.

32. A method of sealing the surface of tissue, the method comprising applying the composition of any one of claims 1 to 16 or claim 31 to the tissue.

33. A method of delivering agents to tissue, the method comprising applying the composition of any one of claims 1 to 16 or claim 31 to the tissue.

34. A composition of any one of claims 1 to 16 or claim 31 for use in sealing the surface of tissue.

35. A composition of any one of claims 1 to 16 or claim 31 for use in delivering agents to tissue.

36. Use of a kit, package or device comprising type I collagen, sodium ions, calcium ions, and one or more crosslinking agents, for preparing a composition comprising:
3-15 mg/ml of the type I collagen;
0.135-5 M of the sodium ions and/or 0.008-0.4 M of the calcium ions; and
the one or more crosslinking agents.

37. The use of claim 36, wherein the composition comprises 0.01-0.5% (w/v) riboflavin or 0.01-0.5% (w/v) Rose Bengal.

38. The use of claim 36 or claim 37, wherein the one or more crosslinking agents are capable of activation by light.

39. The use of claim 38, wherein the light comprises UV light, blue light, green light or white light.

40. The use of claim 36, wherein the composition comprises fibrinogen and thrombin, and wherein
the fibrinogen is present in a first compartment;
the thrombin is present in a second compartment; and wherein
the kit, package or device is configured to allow separation of the fibrinogen of the first compartment and the thrombin of the second compartment during and following loading of the fibrinogen and thrombin into the kit, package or device, and wherein the kit, package or device further comprises a means to facilitate mixing of the fibrinogen of the first compartment with the thrombin of the second compartment.

41. The use claim 40, wherein the composition comprises:
1.6-6 mg/ml fibrinogen; and/or
1-5 U/mL thrombin.

42. The use of any one of claims 36 to 41, wherein the composition further comprises any one or more of: a culture medium, growth factors, hormones, matrix proteins, glycoproteins, vitamins, ions other than sodium ions or calcium ions, ion sources, fibronectin, amino acids, antibiotics, anaesthetics, factor XIII, Fetal Bovine Serum (FBS), human serum, platelet lysate, human platelet lysate.

43. The use of any one of claims 36 to 42, wherein any one or more of the type I collagen, sodium ions, calcium ions and/or one or more crosslinking agents is separated from other component/s within the kit.
